(19) United States
(12) Patent Application Publication
(10) Pub. No.: US 2016/0274120 A1
(43) Pub. Date: Sep. 22, 2016

(54) METHODS OF DETERMINING WHETHER THE WNT SIGNALING PATHWAY IS UPREGULATED IN A TUMOR

(71) Applicant: ICAHN SCHOOL OF MEDICINE AT MOUNT SINAI, New York, NY (US)

(72) Inventors: Stuart Aaronson, New York, NY (US); Gal Akiri, Bronx, NY (US); Sapna Vijayakumar, New York, NY (US)

(73) Assignee: ICAHN SCHOOL OF MEDICINE AT MOUNT SINAI, New York, NY (US)

(21) Appl. No.: 15/078,644

(22) Filed: Mar. 23, 2016

Related U.S. Application Data
(62) Division of application No. 13/825,155, filed on Dec. 15, 2013, now abandoned.
(60) Provisional application No. 61/214,005, filed on Apr. 17, 2009.

(51) Int. Cl. G01N 33/574; C12Q 1/68

(52) U.S. Cl. CPC G01N 33/57484; C12Q 1/6886; C12Q 2600/158; G01N 2333/47; C12Q 2600/118

(57) ABSTRACT

The invention demonstrates that canonical Wnt signaling is activated in certain primary tumors and tumor cell lines in the absence of ?-catenin or APC mutations and that inhibition of such activated canonical Wnt signaling in such tumor cells inhibits tumor growth and, at least in some cases, induces death of tumor cells. As further demonstrated herein, the activation of canonical Wnt signaling is associated with a higher rate of cancer recurrence in patients with Stage I Non-Small Cell Lung Cancer (NSCLC), which provides a new method for cancer prognosis, wherein activation of canonical Wnt signaling reflects a more aggressive tumor phenotype suggesting the need for a more aggressive therapy.

### GOVERNMENT SPONSORED RESEARCH OR DEVELOPMENT

This invention was made with government support under grant 5R01 CA071672 awarded by the National Cancer Institute. The government has certain rights in the invention.

### TECHNICAL FIELD OF THE INVENTION

The present invention relates to methods of cancer diagnosis, treatment and prognosis. Specifically, the invention demonstrates that canonical Wnt signaling is activated in certain primary tumors and tumor cell lines in the absence of β-catenin or APC mutations and that inhibition of such activated canonical Wnt signaling in such tumor cells inhibits tumor growth and, at least in some cases, induces death of tumor cells. As further demonstrated herein, the activation of canonical Wnt signaling is associated with a higher rate of cancer recurrence in patients with Stage I Non-Small Cell Lung Cancer (NSCLC), which provides a new method for cancer prognosis, wherein activation of canonical Wnt signaling reflects a more aggressive tumor phenotype suggesting the need for a more aggressive therapy.

### BACKGROUND OF THE INVENTION

Wnt signaling plays a critical role in cell fate determination and tissue development (Nusse, R. and Varmus, H. E. (1992) Cell 69, 1073-1087; Cadigan, K. M., and Nusse, R. (1997) Genes Dev 11, 3286-3305). Certain members of this family of secreted glycoproteins interact with co-receptors, frizzled (Fzd) and LRP5/6, leading to inhibition of β-catenin phosphorylation by the serine threonine kinase, glycogen synthase kinase-β (GSK-3β) within a large cytoplasmic complex including Dishevelled (Dsh), Adenomatous Polyposis Coli (APC) and Axin (Giles, R. H., van Es, J. H., and Clevers, H. (2003) Biochim Biophys Acta 1653, 1-24). Inhibition of β-catenin phosphorylation impairs its degradation by the ubiquitin/proteasome pathway, resulting in accumulation of the uncomplexed cytosolic molecule. Uncomplexed β-catenin then translocates to the nucleus where it interacts with TCF/LEF, and activates target genes (Giles, R. H., van Es, J. H., and Clevers, H. (2003) Biochim Biophys Acta 1653, 1-24). Accumulating evidence indicates that signaling through the Wnt canonical pathway regulates the differentiation of adult stem cells in the epithelium of the colon (van de Wetering, M., de Lau, W., and Clevers, H. (2002) Cell 109 Suppl, S13-19) and skin (Alonso, L., and Fuchs, E. (2003) Genes Dev 17, 1 189-1200), as well as in muscle (Polesskaya, A., Seale, P., and Rudnicki, M. A. (2003) Cell 113, 841-852) and hematopoietic cells (Reya, T., Duncan, A, W., Ailles, L., Damen, J., Scherer, D. C, Willert, K., Hintz, L., Nusse, R., and Weissman, I. L. (2003) Nature 423, 409-414). Constitutively activated Wnt signaling has also been shown to be causally involved in cancer (Polakis, P. (2000) Genes Dev 14, 1837-1851).

Extracellular inhibitors that function to fine-tune the spatial and temporal patterns of Wnt activity and act at the cell surface to inhibit Wnt signaling through its receptors have recently been discovered (Kawano, Y., and Kypta, R. (2003) J Cell Sci 116, 2627-2634). One group of Wnt antagonists is the secreted Frizzled Related Proteins (FRPs), which share sequence similarity with the Frizzled receptor CRD (cysteine rich domain), but lack the transmembrane and intracellular domains (Leyns, L., Bouwmeester, T., Kim, S. H., Piccolo, S., and De Robertis, E. M. (1997) Cell 88, 747-756; Wang, S., Krinks, M., Lin, K., Luyten, F. P., and Moos, M., Jr. (1997) Cell 88, 757-766; Finch, P. W., He, X., Kelley, M. I, Uren, A., Schaudies, R. P., Popescu, N. C, Rudikoff, S., Aaronson, S. A., Varmus, H. E., and Rubin, J. S. (1997) Proc Natl Acad Sci USA 94, 6770-6775). Through its CRD, FRP exhibits the ability to bind Wnt, form dimers and heterodimerize with Frizzled (Leyns, L., Bouwmeester, T., Kim, S. H., Piccolo, S., and De Roberts, E. M. (1997) Cell 88, 747-756; Wang, S., Krinks, M., Lin, K., Luyten, F. P., and Moos, M., Jr. (1997) Cell 88, 757-766; Rattner, A., Hsieh, J. C, Smallwood, P. M., Gilbert, D. J., Copeland, N. G., Jenkins, N. A., and Nathans, J. (1997) Proc Natl Acad Sci USA 94, 2859-2863; Lin, K., Wang, S., Julius, M. A., Kitajewski, J., Moos, M., Jr., and Luyten, F. P.

(1997) Proc Natl Acad Sci USA 94, 11196-11200; Bafico, A., Gazit, A., Pramila, T., Finch, P. W., Yaniv, A., and Aaronson, S. A. (1999) J Biol Chem 274, 16180-16187). Thus, FRP may act not only to sequester Wnts but also to inhibit Wnt signaling via formation of non-functional complexes with the Frizzled receptor. Another Wnt antagonist is designated Dickkopf-1 (DKK1), which is the prototype of a family of secreted proteins structurally unrelated to Wnt or Frizzled (Glinka, A., Wu, W., Delius, H., Monaghan, A. P., Blumenstock, C, and Niehrs, C.
(1998) Nature 391, 357-362; Fedi, P., Bafico, A., Nieto Soria, A., Burgess, W. H., Miki, T., Bottaro, D. P., Kraus, M. H., and Aaronson, S. A. (1999) J Biol Chem 274, 19465-19472). DKK1 binds the Wnt co-receptor LRP6 and causes its endocytosis through formation of a ternary complex with the transmembrane protein Kremen (Mao, B., Wu, W., Li, Y., Hoppe, D., Stannek, P., Glinka, A., and Niehrs, C. (2001) Nature 411, 321-325; Bafico, A., Liu, G., Yaniv, A., Gazit, A., and Aaronson, S. A. (2001) Nat Cell Biol 3, 683-686; Semenov, M. V., Tamai, K., Brott, B. K., Kuhl, M., Sokol, S., and He, X. (2001) Curr Biol 1 1, 951-961; Mao, B., Wu, W., Davidson, G., Marhold, J., Li, M., Mechler, B. M., Delius, H., Hoppe, D., Stannek, P., Walter, C, et al. (2002 Nature 417, 664-667).

Wnts were initially identified as a consequence of their transcriptional activation by mouse mammary tumor virus promoter insertion, which initiates mammary tumor formation (Nusse, R., and Varmus, H. E. (1992). Cell 69, 1073-1087). Later studies established that genetic alterations afflicting APC and β-catenin, leading to increased uncomplexed β-catenin levels, occur in human colon and some other cancers (Polakis, P. (2000) Genes Dev 14, 1837-1851; Giles, R. H., van Es, J. H., and Clevers, H. (2003) Biochim Biophys Acta 1653, 1-24).

The canonical Wnt/β-catenin pathway plays a key role in the proliferation and differentiation of stem/progenitor cells in a variety of adult epithelial tissues (Clevers, 2006; Reya and Clevers, 2005; van de Wetering et al, 2002). This ability is exploited by cancer cells to promote distinct aspects of self renewal such as survival, proliferation and inhibition of differentiation (Reya and Clevers, 2005). In the same tissues where Wnt signaling normally maintains stem/progenitor cells, constitutive activation of this pathway due to dysregulation or genetic aberrations of key components underlies tumorigenesis. This has been best demonstrated in the intestinal crypt, where Wnt signaling normally regulates the stem cells at the bottom of the crypt (Clevers, 2006; Reya and Clevers, 2005). Aberrant Wnt signaling activation caused by mutations in APC or β-catenin results in uncontrolled expansion of cells that are unable to appropriately differentiate and can ultimately lead to colorectal cancer (CRC) (Clevers, 2006; Klaus and Birchmeier, 2008; Polakis, 2007; Reya and Clevers, 2005). In fact, APC or β-catenin mutations are observed in greater than 90% of CRCs (Morin et al, 1997; Polakis, 2007).

Lung cancer is the most common cause of cancer mortality worldwide for both men and women (Minna et al, 2002). Despite some improvements in therapy over the last 30 years, the prognosis is generally poor with 85-90% patients dying from their disease (Minna et al, 2002). Lung cancers are divided into two histopathologic types, non-small cell lung cancer (NSCLC) and small cell lung cancer (SCLC), which represent approximately 80% and 20% of tumors, respectively (Minna et al, 2002). SCLC have neuroendocrine features and arise mainly from the central airways, while lung adenocarcinomas, the most frequent form of NSCLC, usually originate in the peripheral lung and arise from progenitor cells located in the bronchioles (Clara cells) or alveoli (AT2 cells).

Recent studies have demonstrated the crucial role of Wnt/β-catenin signaling in regulating the balance between normal lung bronchioalveolar stem cells (BASCs) growth and differentiation during early lung development (Reynolds et al, 2008; Zhang et al, 2008). Hyperactivation of β-catenin in lung epithelium of genetically engineered mice leads to defective epithelial differentiation, increased proliferation, expansion of BASCs and can result in lung tumor formation (Mucenski et al, 2005; Okubo and Hogan, 2004; Reynolds et al, 2008; Zhang et al, 2008). In fact, NSCLCs have been reported to exhibit increased levels of cytosolic or nuclear β-catenin as visualized by increased immunostaining (Ohgaki et al, 2004; Shigemitsu et al, 2001). However, mutations of β-catenin or APC, the most common mechanism of aberrant Wnt pathway activation, are relatively rare (Ding et al, 2008; Ohgaki et al, 2004; Shigemitsu et al, 2001; Sunaga et al, 2001). As yet, there has been no systematic investigation of the frequency of functional Wnt pathway activation or the biological effects of its disruption on phenotype of NSCLC or other tumors.

There is a continuing need in the art for the development sensitive and reliable diagnostics and prognostics of cancer and for the development of chemotherapeutic agents useful for treating and preventing cancer. The present invention meets such needs, and further provides other related advantages.

### SUMMARY OF THE INVENTION

In one aspect, the invention provides a method of determining whether a canonical Wnt signaling is activated in a tumor isolated from a subject comprising measuring the amount of uncomplexed β-catenin in the tumor. In a preferred embodiment, the tumor is derived from tissue which has been rapidly frozen after its isolation from the subject. In another preferred embodiment, the level of uncomplexed β-catenin is measured under mild detergent conditions (e.g., a buffer that contains approximately 1% NP-40 or equivalent non-ion detergent). In one embodiment, the uncomplexed β-catenin is captured using a soluble or immobilized E-cadherin protein or a fragment thereof containing β-catenin binding domain. In a specific embodiment, such E-cadherin protein or a fragment thereof is fused to a tag (e.g., GST, His tag or FLAG).

In a specific embodiment, the invention provides a method of determining whether a canonical Wnt signaling is activated in a tumor comprising the steps of (a) preparing a lysate of the frozen tumor tissue sample under mild-detergent conditions, (b) incubating the lysate with soluble or immobilized E-cadherin protein or a fragment thereof containing β-catenin binding domain, (c) isolating the resulting E-cadherin/β-catenin complex, and (d) detecting the E-cadherin/β-catenin complex, In one embodiment, step (d) is performed using an immunoassay (e.g., immunoblotting or ELISA). In a preferred embodiment, at least one of steps (a)-(c) is performed on ice or at less than 4° C.

In another specific embodiment, the invention provides, a method of determining the amount of uncomplexed β-catenin in a frozen tissue sample, comprising (a) preparing a lysate of the frozen sample under mild-detergent conditions, (b) isolating β-catenin from the lysate using GST-E-cadherin beads and (c) detecting the amount of the isolated β-catenin using an immunoassay (e.g., imπranoblotting or ELISA).

In another aspect, the present invention provides a method of determining whether a Wnt signaling is activated in a tumor comprising comparing the level of Axin2 expression in the tumor cells to the level of Axin2 expression in non-tumor normal adjacent tissue cells of the same tissue, wherein an increase in Axin2 expression in the tumor cells as compared to non-tumor normal adjacent tissue cells indicates that the Wnt signaling is activated in the tumor. In one embodiment, Axin2 expression is determined by RT-PCR or expression RNA profiling.

The above methods of the invention can be used, for example, for identifying which tumors would respond to therapies targeted against activated canonical Wnt signaling. Accordingly, the present invention also provides a method for identifying whether a tumor would respond to a therapy targeted against activated canonical Wnt signaling comprising determining whether the canonical Wnt signaling is activated in the tumor using any of the methods of the present invention.

In another aspect, the invention provides a method for cancer prognosis comprising determining whether canonical Wnt signaling is activated in a tumor, wherein activated canonical Wnt signaling indicates a more aggressive tumor phenotype. Activation of the canonical Wnt signaling can be determined using the methods of the present invention or using any other method. In one embodiment, the canonical Wnt signaling is autocrine Wnt signaling. In one embodiment, the tumor does not have genetic alterations of β-catenin and/or APC. In a specific embodiment, the tumor is selected from the group consisting of lung tumors, sarcomas, brain tumors, breast carcinomas, and ovarian carcinomas. In a preferred embodiment, the tumor is Stage I Non-Small Cell Lung Cancer (NSCLC).

In yet another aspect, the invention provides a method for inhibiting growth of a tumor cell characterized by an activated canonical Wnt signaling comprising inhibiting said activated canonical Wnt signaling in said cell. In one embodiment, the tumor cell is characterized by an activated canonical autocrine Wnt signaling. In one embodiment, the tumor cell does not have genetic alterations of β-catenin and/or APC. In a specific embodiment, the tumor cell is derived from a tumor selected from the group consisting of lung tumors (e.g., NSCLC), sarcomas, brain tumors (e.g., gliomas such as, e.g. astrocytoma or glioblastoma), breast carcinomas, and ovarian carcinomas.

In a related aspect, the invention provides a method for killing a tumor cell characterized by an activated canonical Wnt signaling comprising inhibiting said activated canonical Wnt signaling in said cell. In one embodiment, the tumor cell is characterized by an activated canonical autocrine Wnt signaling. In one embodiment, the tumor cell does not have genetic alterations of β-catenin and/or APC. In a specific embodiment, the tumor cell is derived from a tumor selected from the group consisting of lung tumors, sarcomas, brain tumors, breast carcinomas, and ovarian carcinomas. In a preferred embodiment, the tumor cell is derived from a brain tumor (e.g., a glioblasoma or astrocytoma).

In yet another related aspect, the invention provides, a method for sensitizing a tumor cell to a treatment, wherein the tumor cell is characterized by an activated canonical Wnt signaling, comprising inhibiting said activated canonical Wnt signaling in said cell. In one embodiment, said treatment is a chemotherapy (e.g., cisplatin treatment) or radiation treatment. In one embodiment, the tumor cell is characterized by an activated canonical autocrine Wnt signaling. In one embodiment, the tumor cell does not have genetic alterations of β-catenin and/or APC. In a specific embodiment, the tumor cell is derived from a tumor selected from the group consisting of lung tumors (e.g., NSCLC), sarcomas, brain tumors (e.g., gliomas such as, e.g. astrocytoma or glioblastoma), breast carcinomas, and ovarian carcinomas.

### BRIEF DESCRIPTION OF THE FIGURES

FIGS. 1A-1C. Wnt signaling activation in human NSCLC cell lines. (A) 1 mg total cell lysates were subjected to precipitation with a GST-E cadherin fusion protein (Bafico et al, 1998). Total cell lysates (25 μg) and the GST-E-cadherin precipitates were subjected to immunoblot analysis with a mAb directed against β-catenin. (B) FACS analysis, phase contrast and fluorescence images of H460 (upper panel) and H23 (lower panel) NSCLC cells infected with TOP or FOP TCF-GFP reporter lentiviruses or with EGFP expressing lentivirus (LV-GFP). BF—Bright Field, FL—Fluorescence (C) Lentiviral mediated TCF-GFP reporter activity in human NSCLC cells. Results are depicted as the ratio TOP/FOP GFP mean fluorescence intensity (MFI). Results from two independent experiments are shown.

FIGS. 2A-2E. Effects of FRP1 and DKK1 inhibition on Wnt/β-catenin signaling and growth of human NSCLC cells. (A) Effects of constitutive expression of FRP1 and DKK1 on uncomplexed β-catenin in H1819 NSCLC cell line. FRP1 and DKK1 expression was determined by immunoblot analysis as described in Materials and methods. (B) Analysis of NSCLC lines for uncomplexed β-catenin using regulatable expression of HA-tagged FRP1 (upper panel) and Flag-tagged DKK1 (lower panel). NSCLC cells expressing Tet regulatable FRP1-HA or DKK1-Flag were generated as described in supplemental Materials and methods. Expression of FRP1-HA or DKK1-Flag was induced by removal of dox from the culture medium. Cells expressing tetracycline Trans-activator (tTa) were used as control. Analysis of uncomplexed β-catenin was performed as described in Materials and methods using 1 mg total cell lysates, except for A427 cells, where 0.1 mg cell lysate was used. FRP1 and DKK1 expression was determined by immunoblot analysis as described in Materials and methods. (C) FRP1 and DKK1 mediated inhibition of TCF luciferase reporter activity in NSCLC cell lines. Luciferase reporter activity was calculated by dividing the TOP/RL ratio by the FOP/RL ratio. Results were normalized to the results with vector transduced cultures. The values represent the mean±SD from two independent experiments. (D) Real time PCR quantification of FRP1 and DKK1 effects on axin2 mRNA expression. H23, Al 146 and H1819 cells were infected with vector (VEC), FRP1-HA or DKK1-Flag lentiviruses. qRT-PCR was performed as described in supplemental Materials and methods. Relative mRNA expression levels were quantified using the ΔΔC(t) method (Pfaffl, 2001). (E) Effects of DKK1 on cell growth. A549, H23, H1819 and A427 cells were infected with lentiviruses expressing vector (VEC) or DKK1-Flag and 2×10⁴ cells were plated into 60 mm tissue culture dishes. Cultures were visualized using crystal violet staining 2-3 weeks after plating. Expression of flagged tagged DKK1 was assessed by immunoblot analysis as described in Materials and methods.

FIGS. 3A-3E. Overexpression of Wnt2 and Wnt3a contributes to Wnt signaling activation in autocrine NSCLC cells. (A, B) Real time PCR quantification of Wnt2 (A) and Wnt3a (B) expression in H23 and H1819 cells, respectively. To visualize relative expression levels of Wnt2 and Wnt3a, qPCR reactions were removed before saturation and PCR products were separated on 1.5% agarose gel and stained with ethidium bromide. (C) ShRNA knockdown quantification of Wnt2 and Wnt3a. H23 and H1819 cells were infected with lentiviruses expressing shRNA targeting GFP, Wnt2 or Wnt3a. (D) Effect of shRNA knockdown of Wnt2 and Wnt3a on TCF reporter activity. Luciferase reporter activity was calculated by dividing the TOP/RL ratio by FOP/RL ratio. Each column represents the mean±SD of two independent experiments. (E) Effect of shRNA knockdown of Wnt2 and Wnt3a on axin2 mRNA expression. H23 and Hl 819 cells were infected with lentiviruses expressing shRNA targeting GFP, Wnt2 or Wnt3a.

FIGS. 4A-4F. Effects of inducible dominant negative TCF-4 on growth of NSCLC Wnt autocrine cells. (A) Immunoblot analysis of DNTCFs expression. H23 and Hl 819 cells were infected with lentiviruses expressing DNTCF-4 (DN), DN-mOrange (DN-mO) and vector (VEC) under the control of a tetracycline inducible promoter and selected with puromycin in the presence of dox. After washing, cells were divided into separate cell culture dishes in the presence or absence of dox and analyzed by immunoblot 3 days after induction. Expression of DNTCF-4 proteins was detected using an antibody to TCF-4. Lower molecular weight immunoreactive DNTCF species are also observed. Molecular weights in kilodaltons are indicated (kD). Dox—doxycyclin. (B) Effect of DNTCFs on aχin2 mRNA expression. RNA was extracted from H23 and H1819 cells infected as in (A) and maintained in the presence or absence of doxycyclin for 3 days. Dox—doxycyclin. (C) Effect of DNTCFs on cell cycle profile. PI analysis of H23 and H1819 cells infected as in (A) and maintained in the presence or absence of dox at 3 days after induction. Numbers indicate the percentage of cells in G1 or S phase for each cell line analyzed. Results are representative of at least 2 independent experiments. (D) Effects on proliferation of H23 cells expressing inducible DN-mOrange (DN-mO) in the presence or absence of dox, observed at 3 days after induction. BF—Bright field, FL—Fluorescence, Dox—doxycyclin. (E) Effects on growth of H23 and Hl 819 at 2-3 weeks following expression of vector (VEC), DNTCF-4 (DN) and DN-mOrange (DN-mO). 2×10⁴ cells were plated into 60 mm plates in the presence or absence of dox. Cultures were visualized by crystal violet staining. Dox—doxycyclin (F) Effects of DNTCFs on expression of c-Myc, cyclin D1 and p21. Protein lysates from H23 and H1819 cells, infected with vector (VEC), DNTCF-4 (DN) and DN-mOrange (DN-mO) and grown in the presence or absence of doxycyclin for 3 days, were analyzed by immunoblot. Dox—doxycyclin

FIG. 5. Wnt signaling activation in human NSCLC patient samples. Analysis of total and uncomplexed β-catenin in human NSCLC patient samples. Frozen section tissue samples from NSCLC adenocarcinoma tumors and normal adjacent tissues from the same patients were washed twice in PBS. Equivalent aliquots of 300 μg total cell lysates were

subjected to precipitation with a GST-E cadherin fusion protein (Bafico et al, 1998). Total cell lysates (10 μg) and the GST-E-cadherin precipitates were analyzed by immunoblot using a mAb antibody against β-catenin.

FIG. 6. Semi-quantitative RT-PCR Screen for expression of Wnt ligands in NSCLC cell lines. RT-PCR was performed with 5 μg total RNA and amplified with specific primers (Table 4) using One-Step RT-PCR kit. PCR products were run on 1.5% agarose gel stained with ethidium bromide.

FIGS. 7A-7C. Dominant negative TCF-4 (DNTCF) efficiently and specifically inhibits Wnt signaling activation. (A) Effect of DNTCF-4 (DN) and DN-mOrange (DN-mO) on TCF reporter activity. TCF luciferase reporter cell lines were generated by infecting each cell line with TOP or FOP TCF luciferase lentivirus together with renila luciferase (RL) as an internal control for infection efficiency. TOP-RL and FOP-RL cells were infected with viruses expressing vector (VEC), DNTCF-4 (DN) and DN-mOrange (DN-mO). Dual luciferase reporter assay was performed as described in the Materials and methods. Luciferase reporter activity was calculated by dividing the TOP/RL ratio by the FOP/RL ratio. Results were normalized to the results with vector transduced cultures. Each column represents the mean±SD of two independent experiments. (B) Effects of DNTCF-4 and DN-mOrange on growth of NL20 and A549 cells. Mass cultures infected with lentiviruses constitutively expressing vector (VEC), DNTCF-4 (DN) or DN-mOrange (DN-mO) were selected with puromycin, counted and 2×10⁴ cells were plated into 60 mm plates. Cultures were visualized by crystal violet staining after 2 weeks. Equivalent aliquots of lysates from each of the cell lines were immunoblotted for expression of DNTCFs using an anti TCF-4 antibody. Results are representative of 2 independent experiments. (C) Effects of DNTCF-4 and DN-mOrange on growth of H23, Hl 819 and HCC 15 NSCLC cells. Mass cultures infected with lentiviruses constitutively expressing vector (VEC), DNTCF-4 (DN) and DN-mOrange (DN-mO) were selected with puromycin, counted and 2×10⁴ cells were plated into 60 mm plates. Cultures were visualized by crystal violet staining after 2 weeks. Equivalent aliquots of lysates from each of the cell lines were immunoblotted for expression of DNTCFs using an anti TCF-4 antibody. Results are representative of 2 independent experiments.

FIGS. 8A-8D. DNTCFs induce expression of lung differentiation markers in Wnt autocrine NSCLC cells. Quantitative real time PCR analysis of human lung differentiation markers in H23 and H1819 cells following induction of DNTCFs. RNA was extracted from H23 and Hl 819 mass cultures infected with vector (VEC), DNTCF-4 (DN) and DN-mOrange (DN-mO). 50 ng of total RNA from each of the cell lines were subjected to qPCR analysis to quantify the expression of CCSP, AlAT, ICAM-I, MUC-I and TBP. Bars represent relative expression normalized to TBP expression in the same samples. Each column represents the mean of three independent experiments derived from duplicate PCR reactions of the same cDNA±SD. CCSP—Clara cell secretory protein, MUC-I—Mucin 1, cell surface associated, ICAM-I—inter-cellular adhesion molecule 1, AIAT—alpha 1-antitrypsin.

FIGS. 9A and 9B. Activation of canonical Wnt signaling in human astrocytoma cell lines. A.

Western blot showing increased levels of active β-catenin. Briefly, 1 mg of protein lysate was incubated with E-cadherin-GST, followed by pull-down with glutathione sepharose beads. The washed beads were loaded on a polyacrylamide gel, and Western blotting was performed. The blots were probed with a monoclonal β-catenin antibody (Bafico et al, 2004). As a loading control a-tubulin was used. B. Luciferase reporter activity in astrocytoma cell lines. Cancer cell lines with uncomplexed β-catenin were infected with either TOP- or FOP-luciferase constructs. Luciferase activity was measured in these cell lines after 72 hrs and the values normalized to TOP/FOP value in the vector control.

FIGS. 10A and 10B. Activation of canonical Wnt signaling in human sarcoma cell lines. A.

Western blot showing increased levels of active β-catenin. Briefly, 1 mg of protein lysate was incubated with E-cadherin-GST, followed by pull-down with glutathione sepharose beads. The washed beads were loaded on a polyacrylamide gel, and Western blotting was performed. The blots were probed with a monoclonal β-catenin antibody (Bafico et al, 2004). As a loading control a-tubulin was used. B. Luciferase reporter activity in sarcoma cell lines. Cancer cell lines with uncomplexed β-catenin were infected with either TOP- or FOP-luciferase constructs. Luciferase activity was measured in these cell lines after 72 hrs and the values normalized to TOP/FOP value in the vector control. SS:Synovial sarcoma; ES:Ewing's sarcoma

FIGS. 11A and 11B. Activation of canonical Wnt signaling in human osteosarcoma cell lines.

A. Western blot showing increased levels of active β-catenin in tumor cell lines relative to normal human mesenchymal stem cells (hMSC). Briefly, 1 mg of protein lysate was incubated with E-cadherin-GST, followed by pull-down with glutathione sepharose beads. The washed beads were loaded on a polyacrylamide gel, and Western blotting was performed. The blots were probed with a monoclonal β-catenin antibody (Bafico et al, 2004). As a loading control a-tubulin was used. B. Luciferase reporter activity in osteosarcoma cell lines. Cancer cell lines with uncomplexed β-catenin were infected with either TOP- or FOP-luciferase constructs. Luciferase activity was measured in these cell lines after 72 hrs and the values normalized to TOP/FOP value in the vector control.

FIG. 12. Effect of inhibition of Wnt signaling using dnTCF4 on the growth of human osteosarcoma cell lines in vitro. Osteosarcoma cells were infected with lentiviral construct expressing dnTCF4 and selected for 3 days in puromycin. Following selection, cells were plated at 1000 cells/plate density and grown for 14 days. Cells were fixed in 4% formaldehyde and stained with crystal violet.

FIGS. 13A and 13E. DKK1 specifically sensitizes autocrine Wnt NSCLC cells to cisplatin treatment. (A and B) Effects of cisplatin on growth of H23 (A) and A549 (B) cells infected with vector (VEC) or DKK1 expressing lentiviruses. 5×103 H23 and A549 cells expressing vector or DKK1 were plated in 60 mm plates. Cells were treated for 4 hr with 5 or 20 μM cisplatin or saline as control and colonies were visualized 2 weeks later by crystal violet staining. (C and D) Effects of cisplatin on apoptosis of H23 (C) and A549 (D) cells infected with vector (VEC) or DKK1 expressing lentiviruses and treated for 3 days with increasing concentrations of cisplatin. Adherent and floating cells were collected and processed for FACS analysis using annexin/PI. The percentage of annexin positive cells in vector or DKK1 infected cells is represented in the line graph. Results are the mean±SD of 3 independent experiments. Statistical two-way analysis of variance (ANOVA) tests with Bonferroni multiple testing corrections were performed. *−p<0.05, **−p<0.01, ***−p<0.001. (E) Time course analysis of cleaved PARP and casρase-7 in H23 cells infected with vector (VEC) or DKK1 expressing lentiviruses treated with 5 μM cisplatin for 3 days. Cell lysates were collected every 24 hr and analyzed by immunoblot. Molecular weights in kilodaltons are indicated (kD).

FIG. 14. Dot plot analysis of HA235 brain tumor cells uninfected (A), infected with mOrange (B) or DN-mO (C). Cells were harvested 3 days after infection and incubated with Annexin V-APC and analyzed by flow cytometry for mOrange (FL2) and Annexin V-APC. Lower left quadrant-mOrange negative, Annexin V-APC negative cells. Lower right quadrant-mOrange negative, Annexin V-APC positive cells. Upper left quadrant-mOrange positive, Annexin V-APC negative cells. Upper right quadrant-mOrange positive, Annexin V-APC positive cells. Percentage of cells in each quadrant is denoted.

FIG. 15. A graph showing disease-free survival according to Wnt activation in tumors of patients with pathologic stage I NSCLC.

FIGS. 16A-16F. Downregulation of CDC25A, a novel Wnt target gene, inhibits proliferation of human sarcoma cells in vitro. A. CDC25A is a direct target of Wnt signaling in sarcoma cells. Chromatin immunoprecipitation was conducted on DNA extracted from U-2 OS, a Wnt autocrine sarcoma cell line. Monoclonal antibody against β-catenin was used in immunoprecipitation. Axin 2, a known Wnt target gene, was used as a positive control. B. Western blot showing downregulation of Wnt signaling (by the levels of indicated proteins) by dominant negative TCF-4 (dnTCF) in sarcoma cells results in simultaneous decrease in CDC25A expression. C. Downregulation of Wnt signaling (assayed by TCF4 reporter activity) in sarcoma cells expressing dnTCF4. Cells stably expressing TOP luciferase and a normalizer, renilla luciferase, were used in this assay. D. dnTCF expression induces growth arrest in sarcoma cells. Indicated human sarcoma cells were stably infected with dnTCF or an empty vector control and selected in puromycin for 3 days. Cells were plated at 1000 cells/60 mm

density and cultured for 10 days. Cells were fixed using formaldehyde and stained with crystal violet. dnTCF expression did not affect proliferation in a Wnt signaling negative cell line, A 1673, or a low Wnt positive sarcoma cell line, RD. E Knockdown of CDC25A or c-myc induces growth arrest in sarcoma cells. HCTZ 16, a human colon cancer cell line was used for comparison. Indicated cells were stably infected with either empty vector control or shRNA specific for CDC25A or c-myc and selected in puromycin and plated at 1000 cells/60 mm plate and cultured for 10 days. Cells were fixed and stained with crystal violet. F. Western blotting to show specific downregulation of CDC25A or c-myc after shRNA expression in indicated cells.

### DETAILED DESCRIPTION OF THE INVENTION

The present invention is based on developing a new highly sensitive quantitative method for identifying tumors where the canonical Wnt signaling pathway has been activated by detecting uncomplexed β-catenin (i.e., β-catenin within a cell that is not bound to cadherin but is instead free in the cytosol and able to transport to the nucleus to act in concert with TFC/LEF transcription factors to activate TCF target genes). Critical aspects of the method of the invention are (i) the use of freshly and rapidly frozen tissue material (e.g., tissue samples that are snap frozen in liquid Nitrogen in Optimal Cutting Temperature (OCT) compound or not and stored in the same or at −70° C. or lower temperatures), and (ii) the use of mild detergent conditions which allow isolation of uncomplexed β-catenin without disrupting intracellular β-catenin-containing protein complexes and without allowing β-catenin degradation (e.g., buffer that contains approximately 1% NP-40 or equivalent non-ion detergent that solubilizes membrane-associated proteins without disrupting non-covalent protein-protein interactions). This approach makes it possible to solubilize cellular proteins in frozen tissue sections without loss of protein as is critical for quantitative analysis. As shown by the present inventors and co-workers, measurement of uncomplexed β-catenin in tumor cell lines has been shown to correlate with the level of canonical Wnt signaling pathway activation as measured by a transcriptional reporter for TCF/β-catenin (Akiri G, et al., Oncogene. 2009; Bafico A, et al., Cancer Cell 2004) establishing further that this method truly detects pathway activation. As one specific embodiment, the assay of the invention captures uncomplexed β-catenin using a recombinant E-cadherin protein or a fragment thereof containing the β-catenin binding domain, which is fused to a tag allowing for ready isolation and/or detection (e.g., GST, His tag or FLAG) of the resulting E-cadherin/β-catenin complex. In one embodiment, the method of the invention comprises the steps of (a) preparing a lysate of the frozen tissue sample under mild-detergent conditions which allow isolation of uncomplexed β-catenin without disrupting intracellular β-catenin-containing protein complexes and without allowing β-catenin degradation, (b) incubating the lysate with soluble or immobilized E-cadherin or a fragment thereof containing β-catenin binding domain, which is fused to a tag allowing for ready isolation and/or detection (e.g., GST-E-cadherin or His tag-E-cadherin or FLAG-E-cadherin), (c) isolating the resulting E-cadherin/β-catenin complex (e.g., using affinity chromatography or pull-down assay [e.g., with GST beads, Nickel beads, or anti-FLAG mab beads, respectively], or removing lysate if E-cadherin has been pre-immobilized), and (d) detecting the captured uncomplexed β-catenin (e.g., using immunoblot analysis or ELISA).

Various immunodetection assays known in the art can be used for detecting captured uncomplexed β-catenin. Non-limiting examples of particularly useful assays include, e.g., various types of enzyme linked immunosorbent assays (ELISAs), radioimmunoassays (RIA), immunoblot, and immunobead capture assays. In one exemplary ELISA embodiment of the

present invention, E-cadherin protein or fragment can be immobilized onto a selected surface exhibiting protein affinity, such as a well in a polystyrene microtiter plate. Alternatively, antibodies binding to β-catenin or E-cadherin can be immobilized. After the step of uncomplexed β-catenin binding to E-cadherin (or E-cadherin fragment) and washing to remove non-specifically bound immunocomplexes, the bound proteins can be detected. Detection can be achieved by the addition of a second antibody specific for the target protein, that is linked to a detectable label or by the addition of a second antibody, followed by the addition of a third antibody that has binding affinity for the second antibody, with the third antibody being linked to a detectable label.

In conjunction with the detection methods of the present invention, the invention also provides various kits which can be used for detection. For example, a kit can include (a) a soluble or immobilized (e.g., on beads or multiwall plates) E-cadherin or a fragment thereof containing β-catenin binding domain, which is fused to a tag allowing for ready isolation and/or detection (e.g., GST-E-cadherin or His tag-E-cadherin or FLAG-E-cadherin); Qa) tissue solubilization buffer (e.g., containing 1% NP-40), and (c) detection means for detecting captured uncomplexed β-catenin (e.g., anti-β-catenin antibody and a conjugated secondary antibody such as suitable for ECL detection or any other method of signal amplification). A kit can be presented in a pack and may be accompanied by instructions for use.

The present invention also provides a method for determining whether the canonical Wnt signaling pathway is activated in a tumor comprising comparing the level of Axin2 expression in the tumor cells to the level of Axin2 expression in normal non-tumor cells of the same tissue, wherein an increase in Axin2 expression in the tumor cells as compared to normal non-tumor cells of the same tissue indicates that the canonical Wnt pathway is activated in the tumor. Axin2 expression can be determined, e.g., by RT-PCR or expression RNA profiling.

By providing novel methods for detecting whether the canonical Wnt pathway is activated in a tumor, the present invention provides methods for identifying which cancers should respond to therapies targeted against activated Wnt canonical signaling.

Using the methods of the invention, the inventors have identified that canonical Wnt signaling is activated (as demonstrated by increased levels of uncomplexed β-catenin), in the absence of genetic alterations of β-catenin or Adenomatous Polyposis Coli (APC), in several primary tumors and tumor cell lines, including Non-Small Cell Lung Cancer (NSCLC) primary tumors and tumor cell lines, primary sarcomas and sarcoma cell lines of diverse histopathological subtypes, glioblastoma/astrocytoma cell lines, primary human breast and ovarian carcinomas. As further disclosed herein, Wnt2 and Wnt3a overexpression may contribute to activation of canonical autocrine Wnt signaling in NSCLC primary tumors and tumor cell lines. Without wishing to be bound by any theory, it is believed that other canonical Wnt ligands may be also overexpressed in this and other Wnt autocrine tumors, and other mechanisms including LRP5/6 receptor amplification and/or overexpression as well as genetic or epigenetic silencing of Wnt antagonists may occur as well.

The present invention also demonstrates that the activated canonical Wnt pathway is associated with a higher rate of cancer recurrence (including local and distant metastasis) in patients with Stage I NSCLC. Thus, the present invention provides a novel method for cancer prognosis, wherein activated canonical Wnt signaling reflects a more aggressive tumor phenotype, and in this way also provides a method for identifying patients who may benefit from a more aggressive therapy in addition to resection.

Another aspect of this invention is the demonstration that inhibition of activated canonical Wnt signaling in NSCLC, sarcoma and glioblastoma/astrocytoma tumor cells inhibits tumor growth and, at least in some cases, induces death of tumor cells. Specifically, the present invention provides that inhibition of activated canonical Wnt signaling in NSCLC cells inhibits their proliferation and induces a more differentiated phenotype through a mechanism involving c-Myc. The invention further provides that inhibition of activated canonical Wnt signaling in sarcoma cells inhibits their proliferation through a mechanism involving CDC25a. In addition, the invention provides that inhibition of activated canonical Wnt signaling in glioblasoma/astrocytoma cells inhibits their proliferation and also induces apoptosis.

In a more general sense, the present invention provides a method for inhibiting growth of a tumor cell characterized by an activated canonical Wnt signaling, by inhibiting such activated canonical Wnt signaling. Non-limiting examples of encompassed tumor cells include, for example, cells derived from lung tumors (e.g., NSCLC), sarcomas, brain tumors (e.g., gliomas such as, e.g., astrocytomas and glioblastomas), breast carcinomas, ovarian carcinomas, etc.

Any inhibitor of canonical Wnt signaling can be used in the method of the present invention. Such inhibitors include, without limitation, any agent that downregulates expression or activity of any of the elements in a canonical Wnt signaling pathway, including, without limitation, Wnt antagonists, Wnt receptor antagonists, and combinations thereof. Non-limiting examples of useful inhibitors include, e.g., small molecules or blocking antibodies which interact with Wnt or Wnt receptors (e.g., Frizzled) or with Wnt-associated proteins (e.g., LRP5/6, Kremen); soluble Frizzled-related proteins (FRPs such as, e.g, FRP1), which share sequence similarity with the Frizzled receptor CRD (cysteine rich domain), but lack the transmembrane and intracellular domains; Cerberus; Dickkopf (Dkk) proteins (e.g., Dkk-1, Dkk-2, Dkk-3, Dkk-4); Soggy protein (Sgy); Wise; dominant negative TCF-4 (dnTCF4); fusion proteins comprising any of the above; derivatives of any of the above; variants of any of the above; biologically active fragments of any of the above; siRNAs or antisense oligonucleotides which can inhibit expression of any of the elements of an autocrine Wnt signaling pathway (e.g., siRNAs directed against Wnt co-receptors LRP5/6), and any mixtures of any of the above. For a list of useful small molecule inhibitors, see, e.g., www.stanford.edu/˜rnusse/assays/smallmol.html. See also, e.g., Barker N. and Clevers H., Nat Rev Drug Discov. 2006, 5(12):997-1014; Leyns, L., Bouwmeester, T., Kim, S. H., Piccolo, S., and De Robertis, E. M. (1997) Cell 88, 747-756; Wang, S., Krinks, M., Lin, K., Luyten, F. P., and Moos, M., Jr. (1997) Cell 88, 757-766; Finch, P. W., He, X., Kelley, M. J., Uren, A., Schaudies, R. P., Popescu, N. C, Rudikoff, S., Aaronson, S. A., Varmus, H. E., and Rubin, J. S. (1997) Proc Natl Acad Sci USA 94, 6770-6775; Glinka, A., Wu, W., Delius, H., Monaghan, A. P., Blumenstock, C, and Niehrs, C. (1998) Nature 391, 357-362; Fedi, P., Bafico, A., Nieto Soria, A., Burgess, W. H., Miki, T., Bottaro, D. P., Kraus, M. H., and Aaronson, S. A. (1999) J Biol Chem 274, 19465-19472; Gregorieff et al. (2005) Gastroenterology 129:626-638; Krupnik et al. (1999) Gene 238(2):301-13.

While, as specified above, inhibition of canonical Wnt signaling can inhibit tumor growth of tumors where such signaling is activated in the absence of other therapeutic modalities, the present invention also provides that the inhibition of activated canonical Wnt signaling can cooperate with other therapeutic modalities (e.g., chemotheraputics and/or radiation therapy) to enhance tumor cell killing. For example, as disclosed in the Examples section, below, Wnt signaling inhibitor DKK1 specifically sensitizes autocrine Wnt NSCLC cells to cisplatin treatment.

Thus, in a more general sense, the present invention provides a method for sensitizing a tumor cell to a treatment (e.g., chemotherapy or radiation), wherein such tumor cell is characterized by an activated canonical Wnt signaling, comprising inhibiting such activated canonical Wnt signaling. The combination therapy method of the present invention comprises combining inhibiting activated canonical Wnt signaling with any chemotheraputics and/or radiation therapy method useful for a given type of tumor. Examples of chemotherapeutic agents useful in the combination treatments of the invention include, but are not limited to, agents which induce apoptosis, necrosis, mitotic cell death, alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, steroid hormones, and anti-androgens. Some non-limiting specific examples of such useful chemotherapeutic agents include, e.g., cisplatin, erlotinib, Navelbine, gemcitabine (2′-2′-difluorodeoxycytidine), methotrexate, 5-fluorouracil (5FU), taxol, doxorubicin, paclitaxel, mitomycin C, etoposide, carmustine, and Gliadel Wafer.

In the therapeutic methods of the present invention, the Wnt signaling inhibitors can be administered alone or in combination with one or more chemotherapeutic agents and/or radiation treatment to the individual in need thereof, either locally or systemically. Depending on the severity and responsiveness of the cancer to be treated, dosing can be of a single or a plurality of administrations, with course of treatment lasting until cure is effected or diminution of the disease state is achieved. The amount of compounds and radiation to be administered will, of course, be dependent on the subject being treated, the severity of the affliction, the manner of administration, the judgment of the prescribing physician, etc. Evaluation of effectiveness of Wnt signaling inhibition and combination treatments of the present invention can be performed using any method acceptable in the art. For example, for solid tumors, tumor volumes can be measured two to three times a week. Tumor volumes can be calculated using the length and width of the tumor (in millimeters). The effect of the treatment can be evaluated by comparing the tumor volume using statistical analyses such as Student's t test. In addition, histological analyses can be performed using markers typical for each type of cancer.

### DEFINITIONS

As used herein, the term “autocrine Wnt signaling” refers to a situation when Wnt canonical ligands (e.g., Wnt 1, 2, 3, 3A, and 10B) are produced by a cell that contains functional receptors for the same ligands.

The term “canonical” Wnt signaling” refers to a Wnt signaling pathway mediated by β-catenin activation as a transcription factor.

The terms “activated Wnt signaling”, “upregulated Wnt signaling”, “Wnt signaling activation”, and “Wnt signaling upregulation” are used interchangeably to refer to Wnt pathway activation by any mechanism. Pathway activation can be measured, for example, by increased levels of uncomplexed β-catenin in a tumor or tumor cell line, by activation of a TCF/transcriptional reporter in a tumor cell line, or by detection of increased levels of TCF/β-catenin target gene expression (e.g., Axin2) in a tumor or tumor cell line.

The terms “inhibit activated Wnt signaling” and “inhibit upregulated Wnt signaling” refer to any decrease in Wnt signaling activation as measured, for example, by a decrease in TCF transcriptional reporter activity in a tumor cell line, a decrease in the expression level of a Wnt target gene (e.g., Axin2) in a tumor or tumor cell line, or by a decrease in levels of uncomplexed β-catenin in a tumor or tumor cell line.

As used herein, the term “uncomplexed β-catenin” refers to β-catenin within a cell that is not bound to a cadherin but is instead free in the cytosol and able to be transported to the nucleus to act in concert with TFC/LEF transcription factors to activate TCF target genes.

The term “mild detergent conditions” refers to conditions, which allow isolation of uncomplexed β-catenin without disrupting intracellular β-catenin-containing protein complexes and without allowing β-catenin degradation (e.g., buffer that contains approximately 1% NP-40 or equivalent non-ion detergent that solubilizes membrane-associated proteins and other cellular proteins without disrupting non-covalent protein-protein interactions).

As used herein, the term “inhibiting tumor growth” is used to refer to any decrease in the rate of tumor growth and/or in the size of the tumor and/or in the rate of local or distant tumor metastasis in the presence of an inhibitor of the Wnt signaling pathway as compared to the rate of tumor growth and/or in the size of the tumor and/or in the rate of local or distant tumor metastasis in the absence of such inhibitor.

As used herein, the terms “ehemotherapeutic agent”, “chemotherapeutic”, and “chemotherapeutic compound” are used interchangeably and refer to a compound, which is capable of inhibiting, disrupting, preventing or interfering with cell growth and/or proliferation. Examples of chemotherapeutic agents include, but are not limited to, agents which induce apoptosis, necrosis, mitotic cell death, alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, steroid hormones and anti-androgens.

The terms “individual”, “subject”, “patient” and “animal” are used interchangeably to refer to any animal (including humans) that can develop a tumor having an activated canonical Wnt signaling pathway.

The terms “about” or “approximately” are used interchangeably and mean within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition. Cold Spring Harbor, N.Y.: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M J. Gait ed. 1984); Nucleic Acid Hybridization [B. D. Hames & S J. Higgins eds. (1985)]; Transcription And Translation [B. D. Hames & S J. Higgins, eds. (1984)]; Animal Cell Culture [RJ. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932,419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nuc. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218.

### Examples

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

### Canonical Wnt Pathway Activation in Human NSCLC Cell Lines

To investigate Wnt pathway activation in human lung carcinoma, uncomplexed and total β-catenin levels were analyzed in lysates of a large panel of NSCLC and SCLC cell lines as well as 2 immortalized, non-tumorigenic human lung epithelial lines, NHBE and NL20, as controls (Table 3). FIG. IA shows that the majority of NSCLC lines exhibited high levels of uncomplexed β-catenin, reflecting its transcriptionally active form, as detected by a glutathione S-transferase (GST) pull-down assay using recombinant E-cadherin (Bafico et ah, 1998). Most of these lines represented the adenocarcinoma type of NSCLC. In contrast, NHBE and NL20 cells, which showed comparable levels of total β-catenin to these NSCLC tumor lines, demonstrated only very low amounts of uncomplexed β-catenin. Of note, undetectable or very low levels of uncomplexed and total β-catenin were also observed in A549 and H460, two NSCLC cell lines that were previously reported to exhibit Wnt pathway activation (He et al., 2004; You et al, 2004). Of the positives, A427 and HCCl 5 were previously reported to harbor activating mutations in β-catenin (Shigemitsu et al., 2001; Sunaga et al., 2001). Sequencing of β-catenin exon 3 revealed no additional activating β-catenin mutations in any of the other positive tumor lines, In a series of 7 SCLC lines analyzed, no detectable elevation of uncomplexed β-catenin was observed (Table 3), suggesting that Wnt pathway activation is infrequent in SCLC.

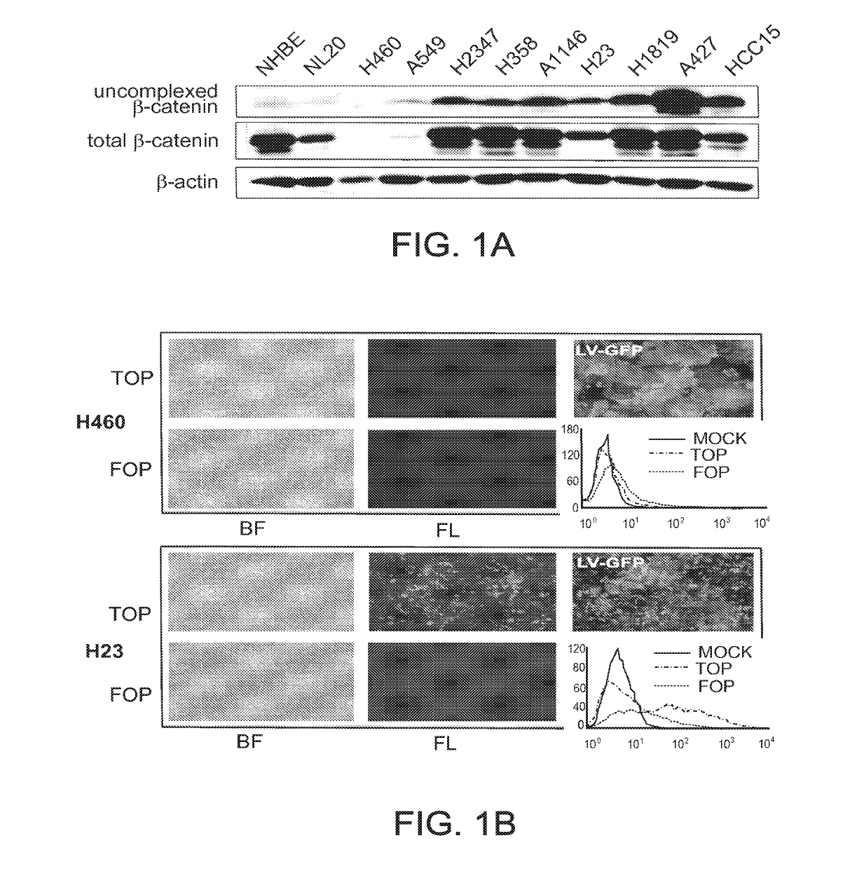

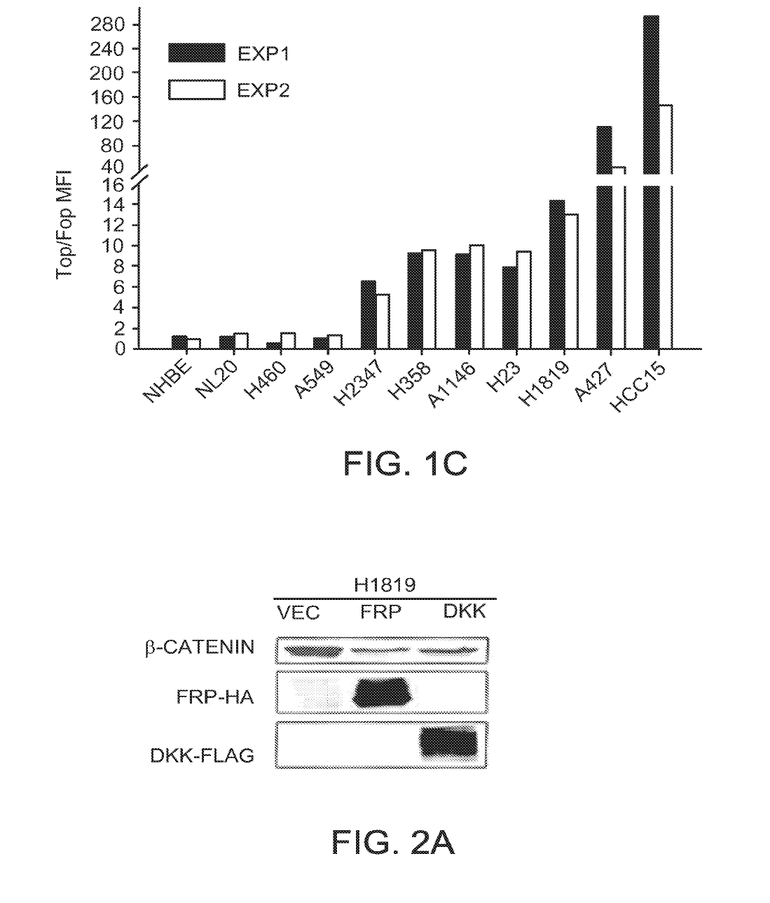

To confirm that the elevated levels of uncomplexed β-catenin observed in NSCLC cells resulted in Wnt pathway activation, a lentiviral-based reporter system for TCF-dependent transcription was developed in which seven wild type (TOP) or mutant (FOP) TCF binding sites (Veeman et al., 2003) were used to drive expression of either EGFP or luciferase. Whereas no activation was seen in H460 cells, H23 cells showed a strong increase in TOP-GFP mean fluorescence intensity (MFI) in comparison to FOP-GFP (FIG. IB). As a control, cell lines were infected with similar efficiency using a lentivirus expressing GFP driven by a constitutive phosphoglycerate kinase (PGK) promoter (LV-GFP). FIG. 1C shows the results of two independent TCF-GFP reporter screens in a series of NSCLC cell lines. Of note, all 4 lines that showed low or undetectable levels of uncomplexed β-catenin (NHBE, NL20, H460 and A549) also showed a low TOP/FOP ratio (less than 2 fold), and were, thus, considered negative for Wnt pathway activation. Using this criterion, elevated levels of TCF-GFP reporter activity were observed in 9 of 16 NSCLC lines (FIG. 1C and Table 3), which generally correlated well with expression levels of uncomplexed β-catenin (FIG. IA).

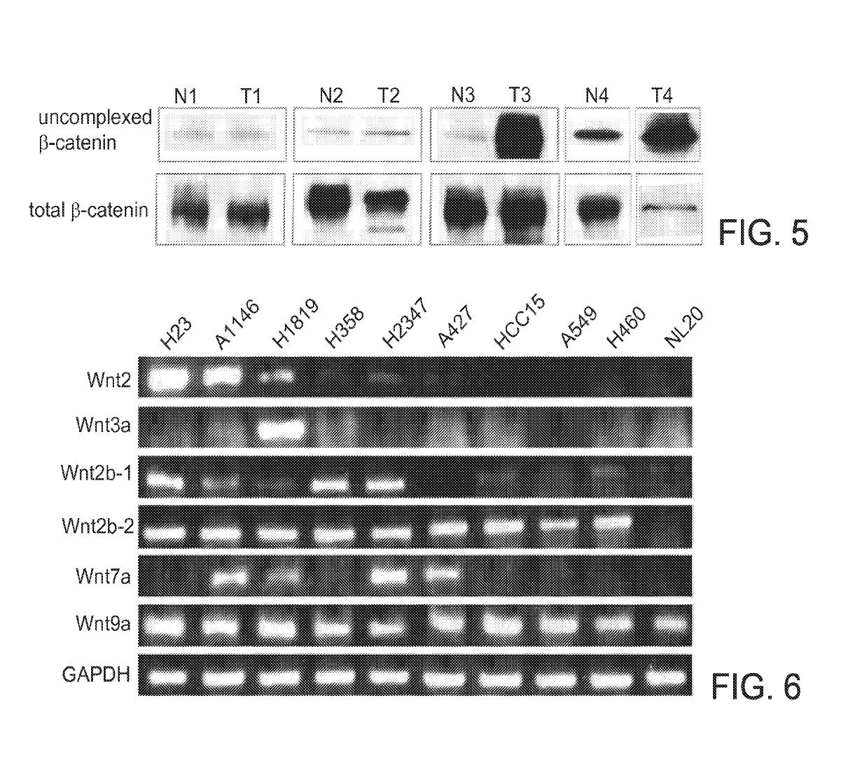

In an effort to extend these findings to primary tumors, five human NSCLCs of the adenocarcinoma subtype were screened for expression of uncomplexed β-catenin. As shown in FIG. 5, out of 3 matched cases of normal and tumor samples from the same patients, two showed similar low levels of uncomplexed β-catenin in both the normal and tumor tissue,

whereas the third tumor showed a striking increase in uncomplexed β-catenin. Increased levels of uncomplexed β-catenin were also observed in two other tumor samples (T4 and T5) as compared to normal lung tissue samples analyzed under identical conditions (N1-N3). Sequencing of the tumor DNAs revealed no detectable β-catenin activating mutations. Collectively, these findings demonstrate increased levels of uncomplexed β-catenin, without genetic alteration of β-catenin, in a high fraction of human NSCLC primary tumors and tumor cell lines.

### Wnt Cell Surface Antagonists Reveal Autocrine Wnt Signaling in Human NSCLC Lines

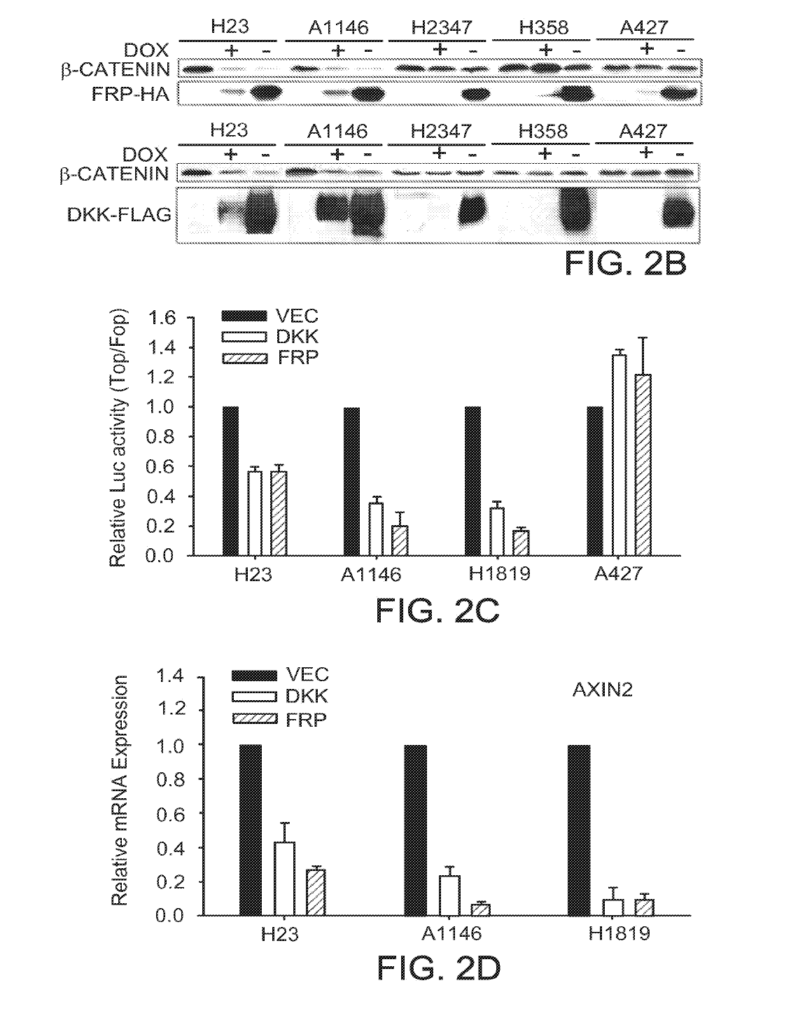

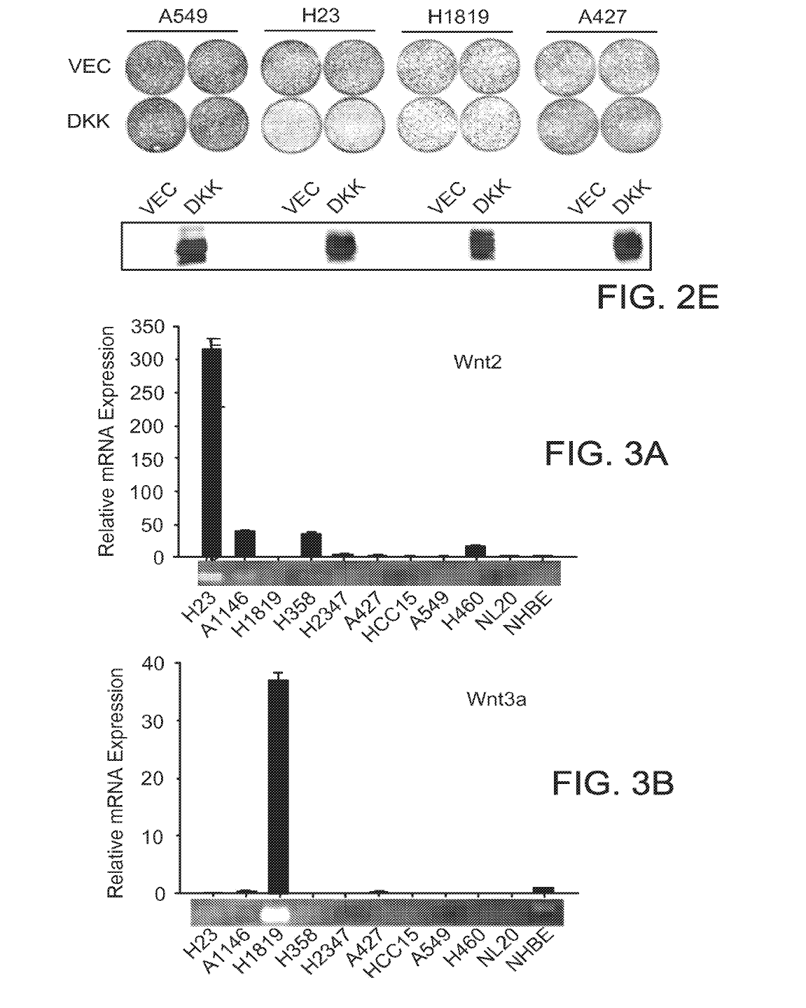

As previously demonstrated by the present inventors, an autocrine mechanism for Wnt pathway activation in human ovarian and breast cancer cell lines associated with high levels of uncomplexed β-catenin in the absence of detectable mutations afflicting either β-catenin or APC (Bafico et ah, 2004). To test the possibility of an autocrine Wnt signaling mechanism in NSCLCs, FRP1 and DKK1 antagonists were used, which inhibit Wnt ligand-receptor interactions (Kawano and Kypta, 2003). Since these inhibitors specifically inhibit Wnt signaling at the cell surface, they can distinguish between extracellular and intracellular pathway activating mechanisms (Bafico et al., 2004). Lentiviral vectors were generated expressing HA-tagged FRP1 or Flag-tagged DKK1 under the control of either a constitutive or Tetracycline (Tet-off) regulatable promoter and tested their ability to decrease the levels of uncomplexed β-catenin and TCF reporter activity. As shown in FIG. 2A, stable expression of FRP1 or DKK1 in Hl 819 NSCLC cells resulted in a marked decrease in uncomplexed β-catenin level. To further explore the effects of FRP1 or DKK1 on uncomplexed β-catenin levels, inducible FRP1 or DKK1 expression in several other NSCLC lines was employed. As shown in FIG. 2B, FRP1 caused a significant reduction in uncomplexed β-catenin levels in H23 and Al 146 tumor lines, even under conditions of low FRP1 expression levels in the presence of doxycycline (dox) due to leakiness

of the inducible system. Similarly, low DKK1 expression levels in the presence of dox were also associated with a decrease in uncomplexed β-catenin levels in H23 and Al 146 cells, which were further reduced upon full induction (FIG. 2A). In contrast, FRP1 or DKK1 showed no effect on uncomplexed β-catenin levels in H2347, H358 or A427 cells, while only A427 cells harbored a β-catenin mutation.

Both antagonists significantly inhibited TCF reporter activity in Hl 819, H23 and Al 146 tumor lines, corroborating the observed decrease in uncomplexed β-catenin levels (FIG. 2C). In contrast, TCF reporter activity in A427 cells was unaffected by these antagonists consistent with their lack of effects on β-catenin levels in these cells (FIG. 2A). As shown in FIG. 2D, each antagonist also significantly downregulated expression of axin2, a prototypic Wnt target gene (Jho et al, 2002), in Hl 819, H23 and Al 146 tumor lines. Taken together, these results demonstrate activation of canonical autocrine Wnt signaling in these NSCLC lines. Conversely, the lack of effects of these Wnt antagonists on H2347 and H358 tumor lines, which showed increased uncomplexed β-catenin levels and increased TCF reporter activity in the absence of β-catenin mutations, implies that other mechanisms were responsible for Wnt signaling activation in these NSCLC lines.

Wnt signaling promotes proliferation and altered cell growth properties (Bafico et al, 1998). To study the effects of Wnt pathway inhibition on tumor cell proliferation, DKK1 was stably expressed in several NSCLC cell lines. As shown in FIG. 2E, DKK1 exerted antiproliferative effects on H23 and H1819 tumor cells in comparison to vector control cells. To confirm that these effects were due to Wnt activity inhibition, the effects of DKK1 on A549 cells, which showed no evidence of Wnt pathway activation, were compared to the effects of DKK1 on β-catenin mutant A427 cells. As expected, expression of DKK1 in these NSCLC lines was not associated with any detectable growth inhibition. Similar expression levels of Flag-tagged DKK1 were confirmed in each of these cell lines by immunoblot analysis (FIG. 2E). Taken together, these results strongly support a role of canonical autocrine Wnt pathway activation in promoting the proliferation of Wnt autocrine NSCLC cells.

### Identification of Canonical Wnts Involved in Autocrine Activation of Human NSCLC Lines

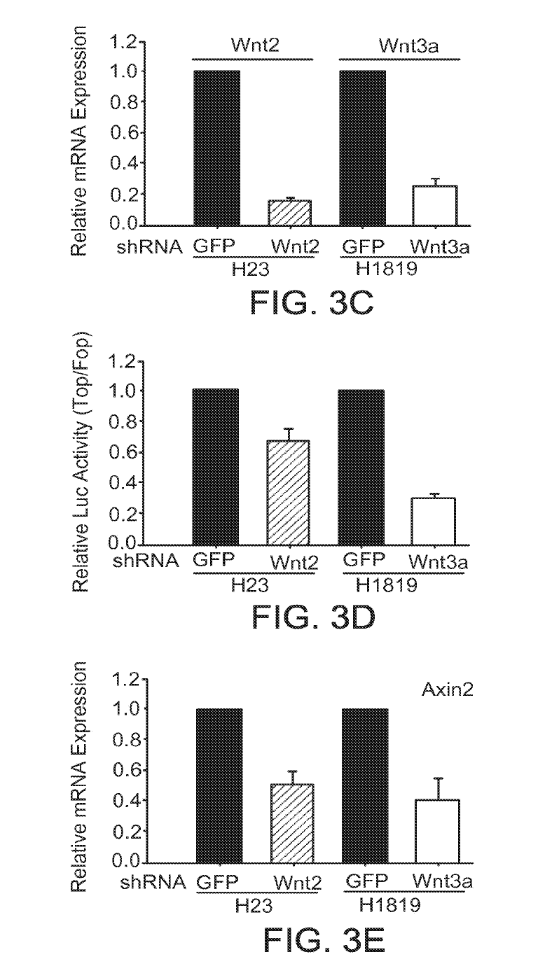

The next goal was identification of Wnt ligands, which might be overexpressed in these tumor cells. Semi-quantitative RT-PCR for expression of 19 Wnts revealed that some were ubiquitously expressed in both β-catenin positive and negative NSCLC lines (Wnt2b, Wnt7a and Wnt9a), whereas Wnt2 and Wnt3a mRNAs were overexpressed primarily in the tumor lines exhibiting autocrine signaling (FIG. 6). No other canonical or non-canonical Wnts were detected using this method. Real-time PCR was utilized to more accurately quantitate Wnt2 and Wnt3a expression levels in a panel of 9 NSCLC and the immortalized NHBE and NL20 lines. FIGS. 3 A and B show that Wnt2 mRNA expression levels in H23 and Al 146 cells were more than 300 fold and 30 fold, respectively, above that of NHBE or NL20 cells, and Wnt3a expression in Hl 819 tumor cells was almost 40 fold higher than in NHBE cells.

To establish whether these Wnts played a role in the activation of canonical autocrine signaling in H23 and H1819 cells, an shRNA knock down approach was utilized. As shown in FIG. 3C, Wnt 2 and Wnt3a shRNA constructs efficiently knocked down the expression of their corresponding mRNA targets (80% for Wnt2 and 70% for Wnt3a). Knockdown of Wnt2 in H23 cells and Wnt3a in Hl 819 cells resulted in each case in a significant decrease in both TCF mediated Iuciferase reporter activity and axin2 expression (FIG. 3D, E). These results provide strong evidence that the activation of canonical autocrine Wnt signaling in H23 and H1819 NSCLC cells involves Wnt2 and Wnt3a, respectively.

Dominant Negative TCF-4 Induces p21 Associated Cell Cycle Arrest of NSCLC Cells with Canonical Wnt Pathway Activation

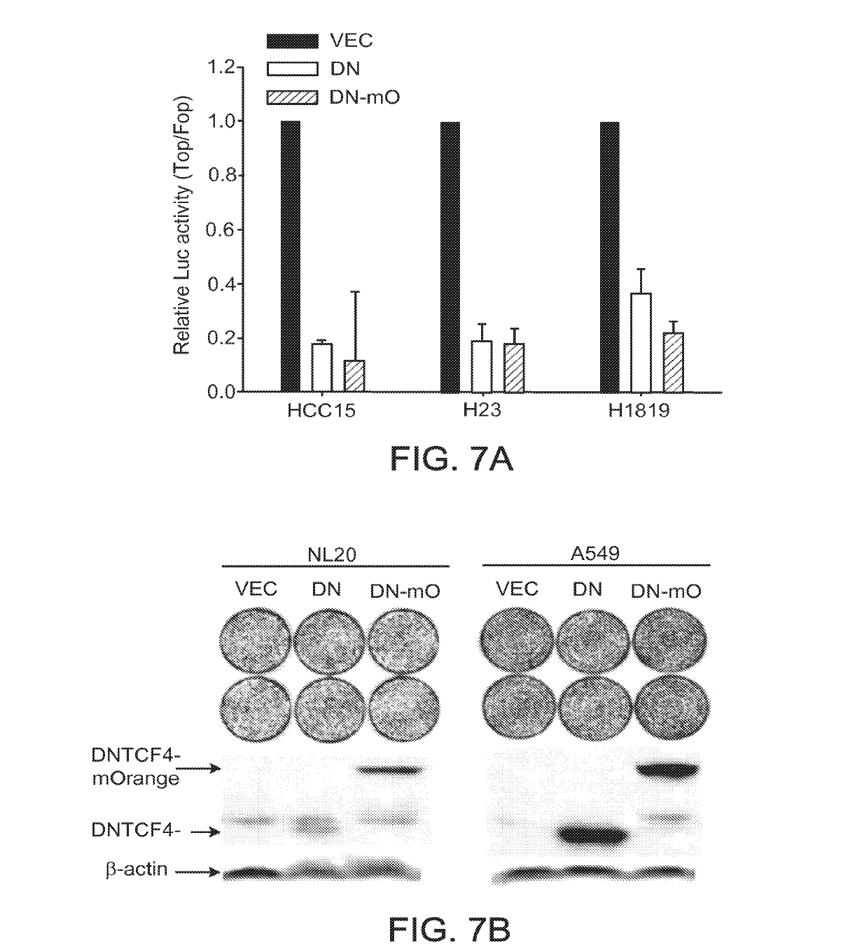

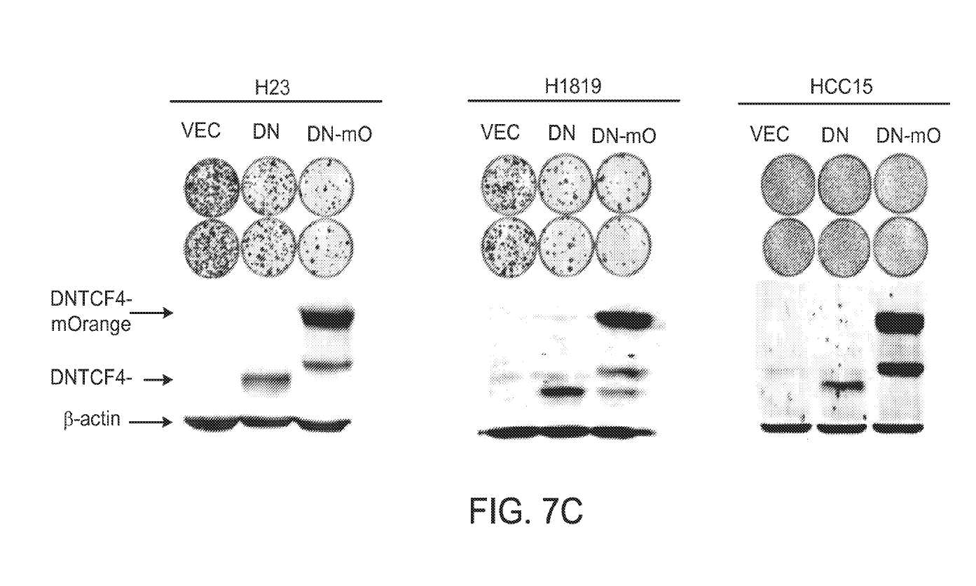

To compare the biological effects of Wnt pathway downregulation in NSCLC lines with Wnt autocrine and β-catenin activating mutations as well as mechanisms involved, constitutive or Tet regulatable expression of a dominant negative TCF-4 (DNTCF-4), which lacks the first 32 amino acids and is unable to bind β-catenin but retains its DNA binding ability, was utilized (van de Wetering et al, 2002). This approach was used previously to investigate the effects of Wnt pathway inhibition in colon carcinoma cells with APC or β-catenin oncogenic mutations (van de Wetering et al., 2002). Lentiviral constructs expressing two versions of either constitutive or inducible DNTCF-4 (designated DN) or DNTCF-4 fused to mOrange (designated DN-mO) were generated. To assess the ability of DN and DN-mO to inhibit Wnt activation, their effects on TCF luciferase reporter activity were tested. As shown in FIG. 7A, both DNTCFs strongly inhibited the constitutively high levels of TCF reporter activity in HCC 15 tumor cells harboring a β-catenin mutation, as well as in Wnt autocrine H23 and Hl 819 NSCLC lines. FIG. 7B shows that DNTCFs expression exerted no effect on the growth of NL20 or A549 cells without any detectable Wnt pathway activation (FIG. 1). In contrast, constitutive expression of both DNTCFs resulted in obvious growth inhibition of Wnt autocrine H23 and H1819 tumor cells (FIG. 7C). DN-mO was more potent, presumably because the DNTCF-4 mO fusion product exhibited an extended half-life (FIG. 3SC). DN-mO also inhibited to a lesser extent the growth of HCC15 cells, which contained a β-catenin mutation (FIG. 3SC).

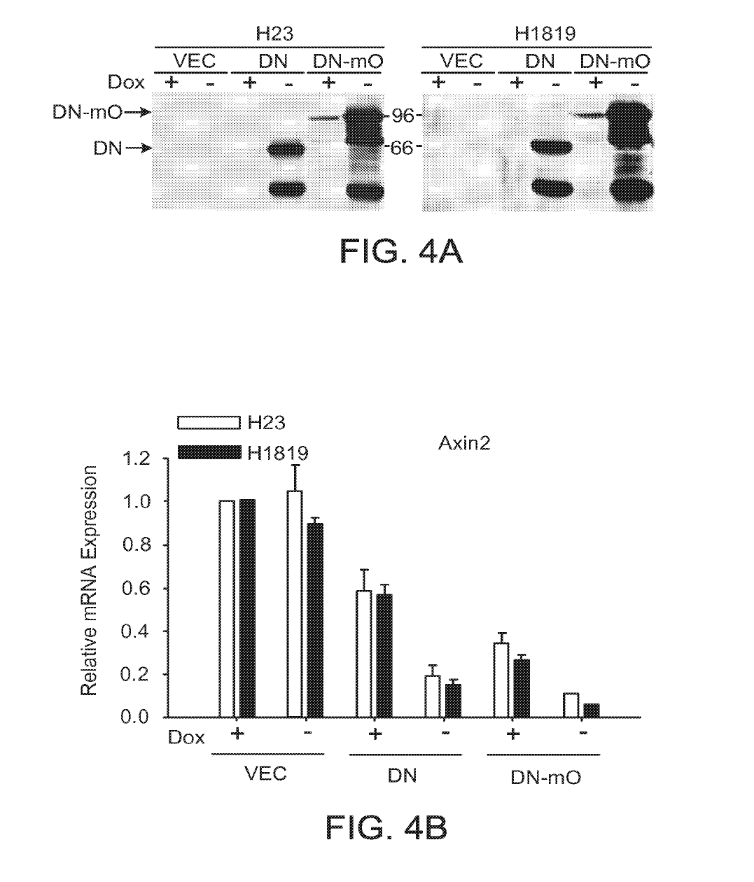

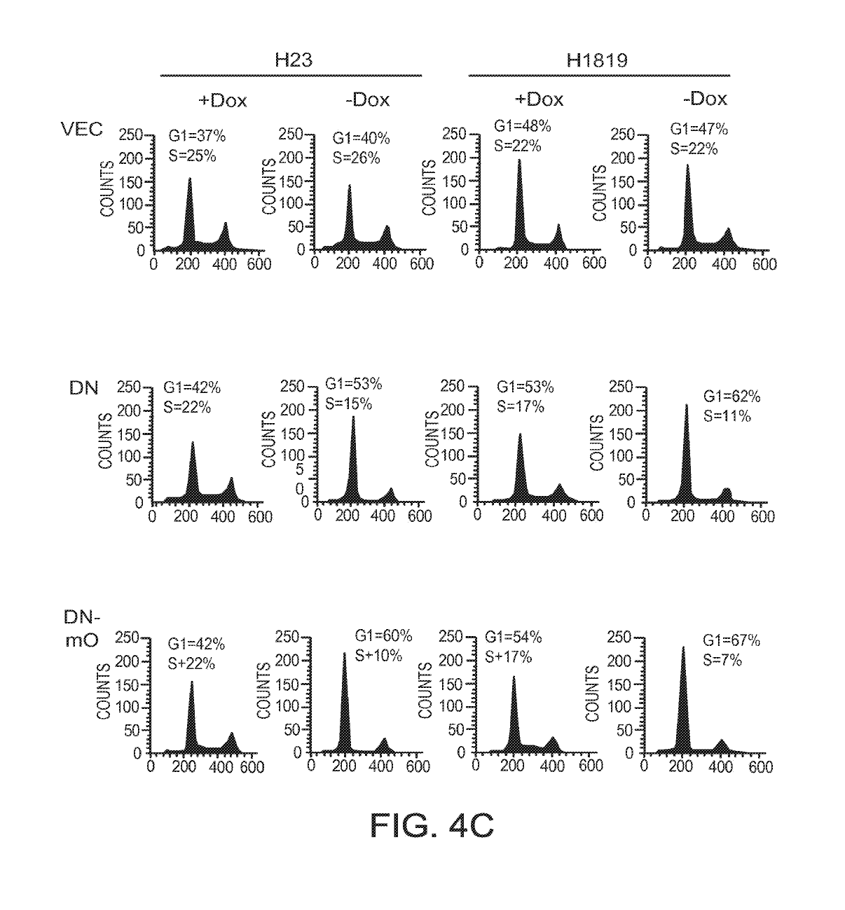

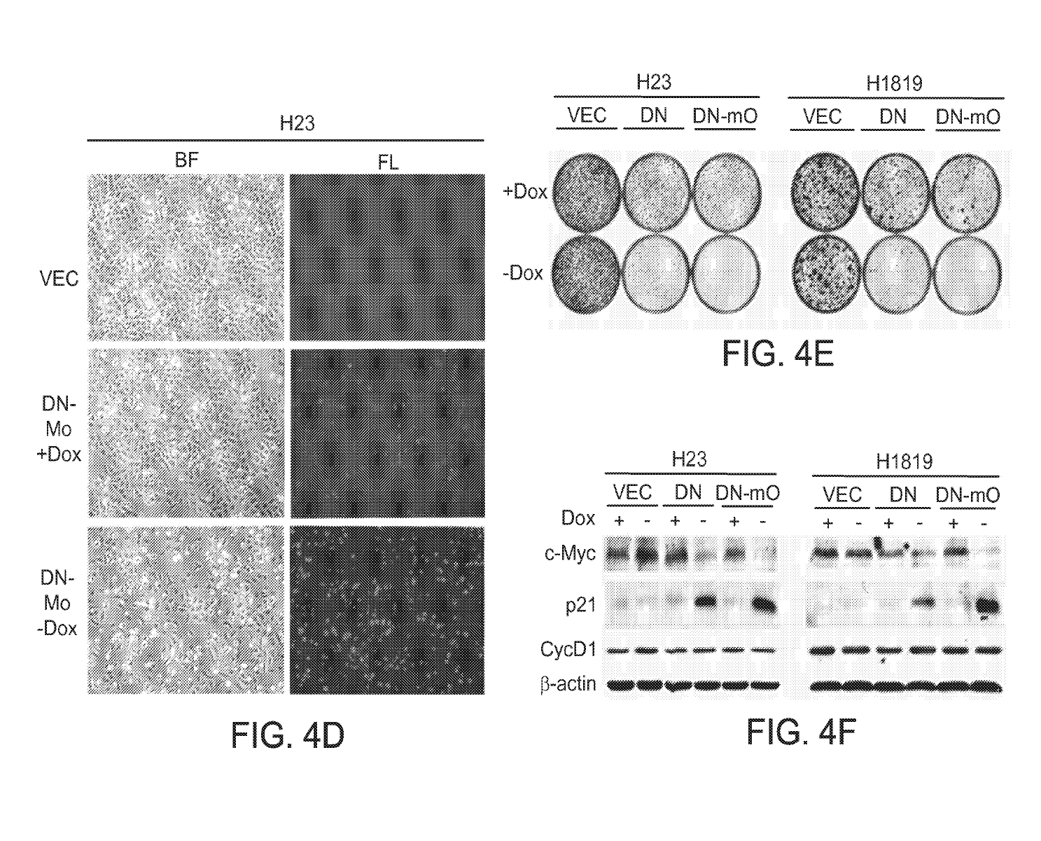

To further investigate the effects of DNTCF expression, mass populations of H23 and H1819 cells expressing Tet inducible versions of DNTCF-4 and DN-mOrange as well as control lentivector (designated VEC) were established. Low expression levels of the two DN forms were observed even in the presence of dox due to leakiness of the system (FIG. 4A), and strong induction was observed upon dox removal. To determine the extent to which the low and high DNTCF expression levels inhibited Wnt pathway activation in H23 and Hl 819 cells, expression of axin2 (Jho et al, 2002) was analyzed by real time PCR. As shown in FIG. 4B, even low expression levels of the DN forms in the presence of dox were sufficient to cause some reduction in axin2 mRNA expression levels. Full induction of the two DN forms resulted in further reduction in levels of this prototypic Wnt target gene. While leaky expression of the two DN forms in the presence of dox retarded cell growth, full induction of DN or DN-mO resulted in strong G1 arrest as measured by FACS at 72 hours. Of note, there was no detectable increase in apoptosis under the same conditions (FIG. 4C). An example of the expression of DN-mO in the presence or absence of dox, and its effects on proliferation of H23 cells is shown in FIG. 4D. As shown in FIG. 4E, leaky expression of the DNTCFs in the presence of dox was associated with decreased colony forming ability as compared to VEC cells. However, full induction of DN and DN-mO exerted more profound growth inhibition.

It was previously shown that inhibition of TCF signaling in clonally selected human CRC lines with APC or β-catenin mutations by regulatable expression of DNTCF-4 induced cell cycle arrest associated with decreased expression of c-Myc and increased expression of the cell cycle inhibitor, p21 (van de Wetering et al., 2002). It was also shown that c-Myc normally inhibits p21 transcription, so that reduced c-Myc expression resulting from TCF signaling inhibition releases p21 to mediate cell cycle arrest and differentiation effects in these cells (van de Wetering et al., 2002). Thus, expression levels of these cell cycle regulators were analyzed by immunoblot analysis in NSCLC cells expressing DNTCFs or VEC in the presence or absence of dox. As shown in FIG. 4F, leaky expression of the two DNTCFs in mass cell populations decreased c-Myc expression, especially with the more potent DN-mOrange, and full induction led to a dramatic inhibition of c-Myc expression. Upon full DNTCF induction, strong upregulation of p21 levels was observed as well. Cyclin D1 protein levels were not significantly affected by the expression of either form of DNTCF-4. The reduction in c-Myc expression levels with concomitant increase in p21 correlated well with cell cycle profile analysis and the effects observed on cell growth (FIGS. 4C-F). Taken together, these findings in NSCLC cells strongly support previous findings that c-Myc is a key mediator of cell proliferation induced by Wnt signaling through a mechanism involving p21 repression (van de Wetering et al, 2002).

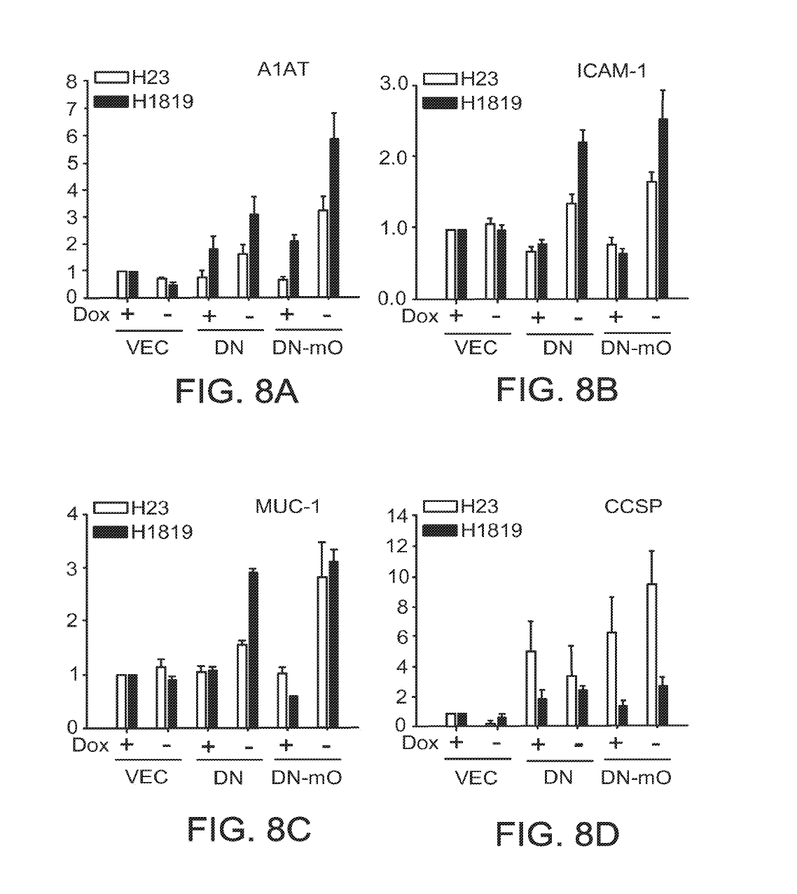

Canonical Wnt signaling has been shown to maintain lung epithelial cells in an undifferentiated stem/progenitor like state (Reynolds et al, 2008; Zhang et al, 2008). Thus, it was also examined whether expression of DNTCF in H23 and Hl 819 cells altered their differentiation state. Real time PCR analysis showed that induction of DN and DN-mO led to an upregulation of several differentiation markers known to be expressed in differentiated bronchiolar (CCSP) or alveolar type 2 (AT2) cells (AlAT, ICAM-I and MUC-I) (FIG. 8) (Braga et al, 1992; Guzman et al, 1994; Nakamura et al, 2006; Venembre et al, 1994). These results demonstrate that inhibition of autocrine Wnt signaling by DNTCF-4 leads to increased expression of differentiation markers associated with both alveolar (AT2) and bronchial (Clara) lineages.

### Discussion

The present findings establish that canonical Wnt signaling activation, as demonstrated by increased levels of uncomplexed β-catenin, occurs at high frequency in NSCLC cell lines and in primary NSCLCs. Upregulated TCF reporter activity was found to generally correlate well with increased levels of uncomplexed β-catenin and provided confirmation of canonical Wnt pathway activation in the tumor lines. Of 9 positive NSCLC lines, only two contained mutations in β-catenin, the most frequently reported genetic aberration in tumors other than CRC, where

APC loss of function mutations are generally more prevalent (Clevers, 2006; Polakis, 2007). The presently observed high incidence of Wnt signaling activation in NSCLCs, in the absence of genetic alterations of β-catenin or APC, argues that this pathway is a much more frequent target than has been previously recognized in this common epithelial tumor. The present findings were also specific to NSCLC as a survey of human SCLC lines revealed no evidence of Wnt pathway activation in this type of lung cancer.

Wnt antagonists, FRP1 and DKK1, which inhibit Wnt signaling at the cell surface (Kawano and Kypta, 2003), caused dramatic decrease of uncomplexed β-catenin levels, TCF reporter activity and expression of the prototypic Wnt target gene axin2 in around 30% of Wnt activated NSCLC lines, strongly implicating a Wnt autocrine mechanism. It was observed further that either Wnt2 or Wnt3a were specifically overexpressed and that their specific shRNA knockdown decreased TCF reporter activity and axin2 expression in these tumor lines. The Wnt2 gene resides on the long arm of chromosome 7 in proximity to a number of proto-oncogenes including c-MET, which can be amplified in lung tumors. However, real time PCR analysis of H23 and H1819 cells showed no evidence of either Wnt2 or Wnt3a gene amplification in these Wnt autocrine tumor lines. Hence, the underlying mechanism responsible for the specific overexpression of either Wnt2 or Wnt3a in enforcing a Wnt autocrine loop in NSCLCs remains to be elucidated.

Previous reports have suggested autocrine Wnt signaling activation in certain NSCLC lines in which either Wnt1 or Wnt2 expression was detected by antibodies, which could also induce the same tumor cells to undergo apoptosis (He et al., 2004; You et al., 2004). These studies chiefly focused on A549 and H460 tumor lines, in which no detectable expression of Wnt1 was observed, and Wnt2 was also undetectable in A549 cells by sensitive RT-PCR techniques. Of note, these tumor lines exhibited only very low or undetectable levels of uncomplexed as well as total β-catenin and also lacked evidence of upregulated TCF reporter activity. Moreover, no detectable biological effects of known Wnt antagonists or DNTCF on A549 cells were observed. In contrast, the Wnt autocrine NSCLC lines identified herein exhibited growth inhibition in the absence of detectable apoptosis in response to these same inhibitors under conditions in which they also caused downregulation of β-catenin and TCF reporter activity. Of note are prior results in CRC (van de Wetering et al, 2002) and breast/ovarian tumor cells (Bafico et al, 2004), respectively, where downregulation of Wnt signaling resulted in cell growth inhibition rather than apoptosis. Thus, the results of He et al. (2004) and You et al. (2004) with A549 (Giard et al, 1973) as well as with H460 unlikely reflect a mechanism involving activated Wnt signaling.

Several Wnt pathway activated NSCLC lines were identified, which exhibited no detectable evidence of Wnt signaling inhibition by either DKK1 or FRP or β-catenin mutations. Without wishing to be bound by any theory, the present findings imply the existence of at least three distinct mechanisms that together account for the high frequency of canonical Wnt activation in human NSCLCs.

Recent studies suggest that BASCs may be the cells of origin of murine lung adenocarcinoma (Kim et al., 2005). Notably, BASCs show Wnt signaling activation (Zhang et al, 2008) and can give rise to progeny with either Clara cell or AT2 cell phenotype (Kim et al, 2005). The cell cycle arrest induced by DNTCF-4 initiated a differentiation program towards both Clara (CCSP) and AT2 (AIAT, ICAM-I and MUC-I) cell lineages. This suggests that a high proportion of human adenocarcinomas may originate from Wnt positive BASCs or, alternatively, that aberrant activation of Wnt signaling in more differentiated progenitors, may endow them with stem/progenitor properties including enhanced proliferative capacity and cell survival properties.

The present showing that downregulation of TCF signaling in autocrine NSCLC cells resulted in decreased c-Myc levels concomitant with increased p21 expression. These findings are consistent with the observed effects of TCF downregulation on c-Myc and p21 levels in CRC lines mutant for APC or β-catenin (van de Wetering et al., 2002). In fact, the oncogenic effects conferred by loss of APC on the mouse small intestine were shown to be almost entirely dependent on functional c-Myc as simultaneous deletion of APC and c-Myc rescued the APC knockout tumor phenotype (Sansom et al, 2007). Of note, c-Myc is frequently overexpressed in lung cancer (Richardson and Johnson, 1993). Although gene amplification can explain its deregulation in a subset of tumors and cell lines, c-Myc overexpression is seen in a much higher percentage of cases in the absence of gene amplification (Bernasconi et al, 2000; Richardson and Johnson, 1993). Thus, the high prevalence of Wnt pathway activation observed herein NSCLC cell lines and primary tumors may help to account for the high frequency of c-Myc overexpression in NSCLC.

### Biochemical Assay for Detection of Canonical Wnt Pathway Activation in Tumor Samples.

The only presently available method to test whether the canonical Wnt signaling pathway is activated in a tumor is to immunostain fixed tissue for β-catenin. Unfortunately, this method is both insensitive and subjective as normal and tumor cells typically express high levels of β-catenin, and the observer must distinguish β-catenin present in cytosol or nucleus, where it is not normally expressed against the background of β-catenin associated with cell membrane bound to cadherins where it is present at much high levels. This is difficult with either formalin fixed tissue or frozen sections. While antibodies generated against hypo-phosphorylated β-catenin have been reported to detect the active form of the molecule, these antibodies are controversial as to their specificity as well as sensitivity in immunostaining. Some of these approaches may be of applicable when uncomplexed β-catenin is markedly increased due to certain mutations such as APC mutations, but they are not sensitive enough to detect activation by autocrine Wnt and other mechanisms discovered by us. For example, in one representative report involving immunostaining for β-catenin in lung tumors cited as high as 87% exhibiting cytoplasmic staining which the authors indicated was not helpful as it contained almost the entire cohort. Further, only a minority of tumors showing either only nuclear or membrane staining could be segregated for any attempt at meaningful analysis (Kotisinas et al. Am J. Path. 2002 161 p. 1619-1634). These authors were unable to identify a relationship even in this small subset of patients with survival.

A novel approach identified by the present invention overcomes these problems by using freshly-frozen and stored tumor tissue, which when gently disrupted in the cold (i.e., on ice or at less than 4° C.) using chilled mortar and pestle to disrupt and homogenized with mild detergent, or using frozen sections not requiring even mortar and pestle, preserves membrane bound β-catenin without releasing it and confounding measurement of uncomplexed β-catenin, the active form of β-catenin, which can serve as a heterodimeric transcription factor in concert with TCF/LEF transcription factors. The uncomplexed β-catenin in tumor samples prepared in this manner can be detected by a GST-E-cadherin capture assay.

The present disclosure establishes efficacy of this assay, its sensitivity for detection in a wide array of tumor types including lung, sarcomas of various types, and primary brain tumors. Importantly, some tumors are negative in this test as are normal tissues prepared and tested under the same conditions. The following table (Table 1) identifies examples of human tumors tested using this frozen-tissue GST-E-cadherin capture assay to determine whether the Wnt signaling pathway was activated in these tumors.

Table 1. Examples of human tumors showing upregulated Wnt signaling

| Tumor sample | Positive for free beta-catenin |
|---|---|
| Fibrosarcoma | 1/1 |
| Liposarcoma | 1/6 |
| Osteosarcoma | 1/1 |
| Chondrosarcoma | 1/2 |
| Rhabdomyosarcoma | 1/1 |
| Ewing's sarcoma | 1/2 |
| Leiomyosarcoma | 0/1 |
| GIST | 1/1 |
| Ovary | 5/6 |
| Glioblastoma | 1/1 |
| Normal tissue | 0/2 |

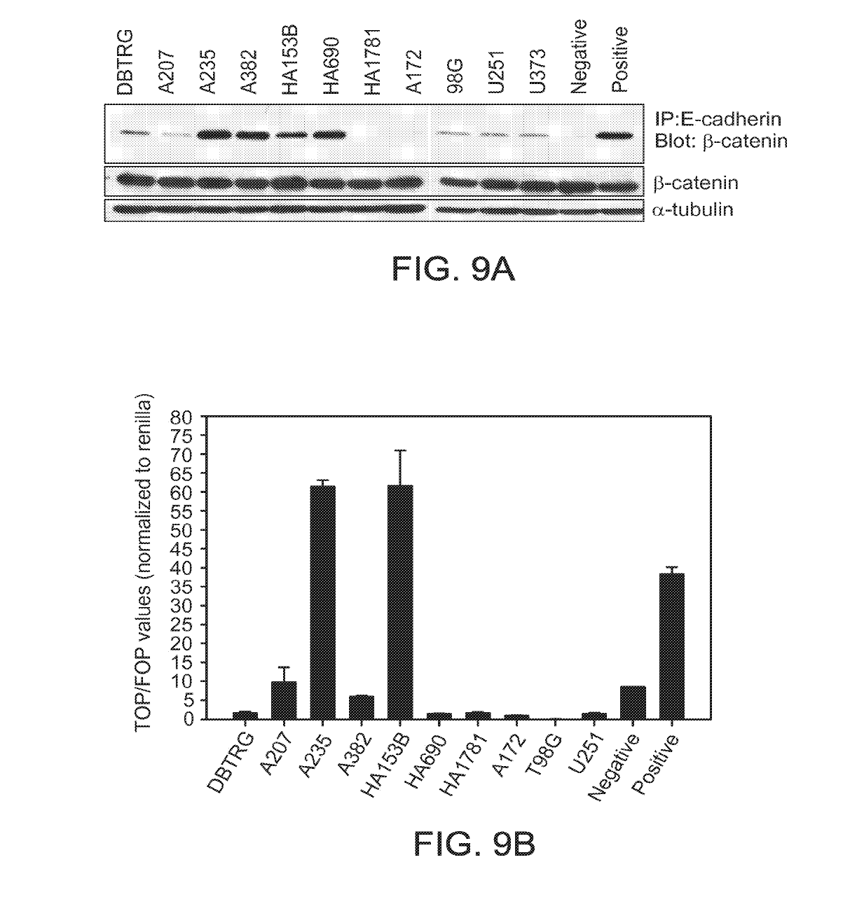

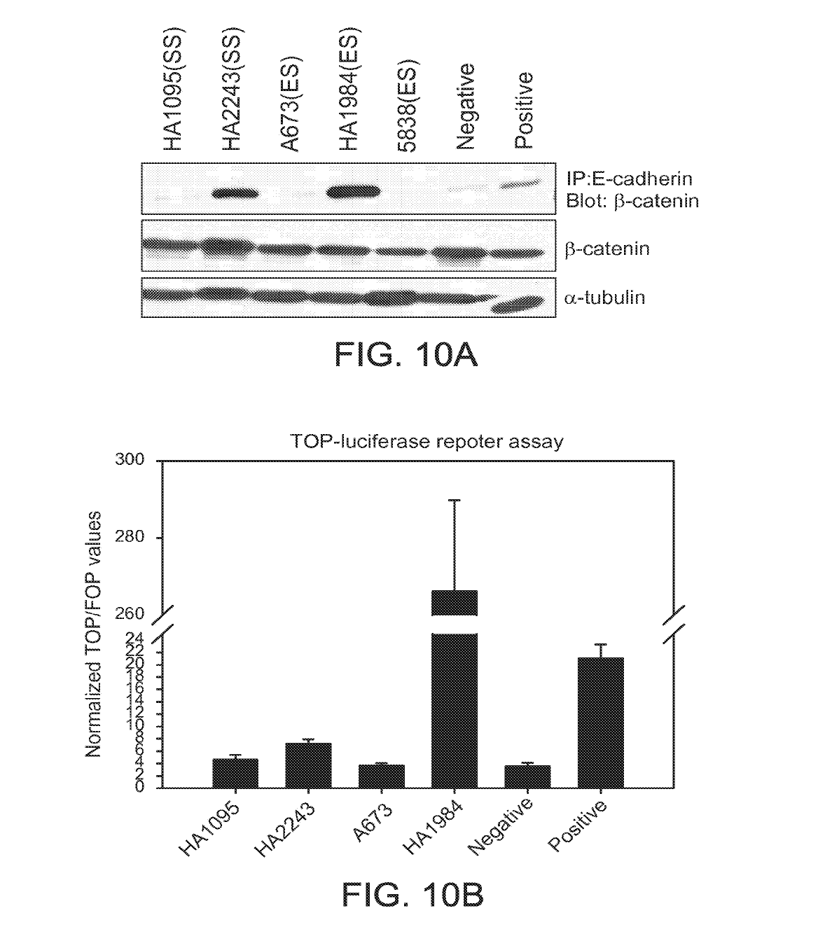

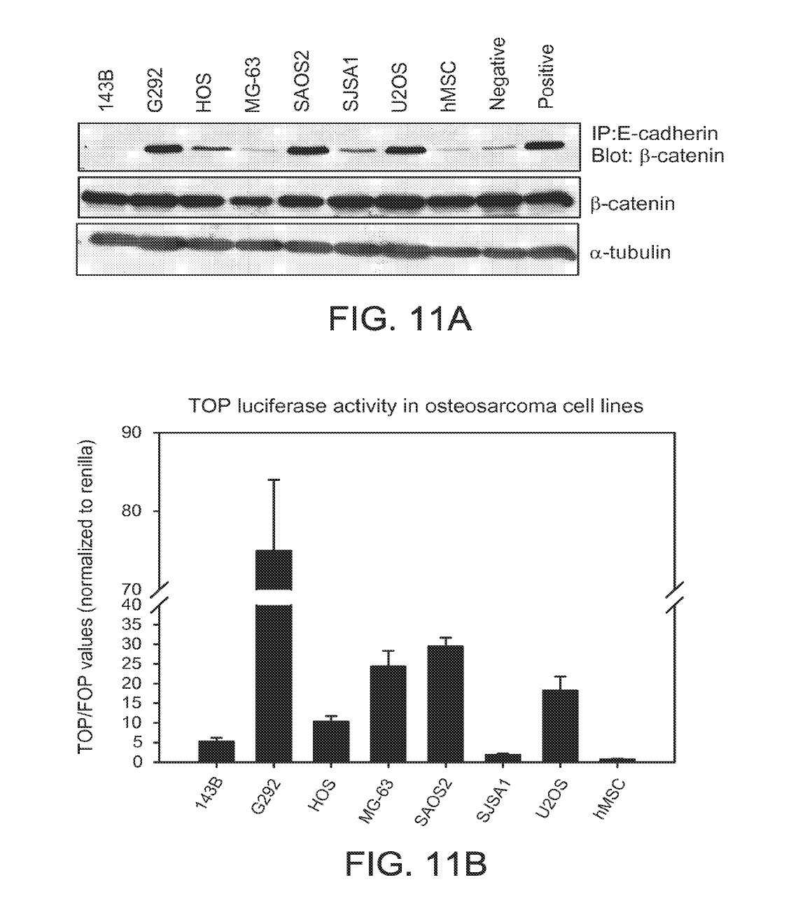

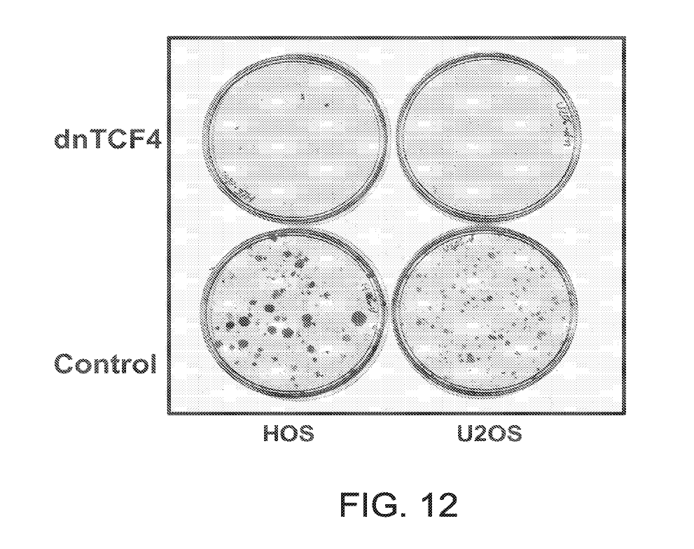

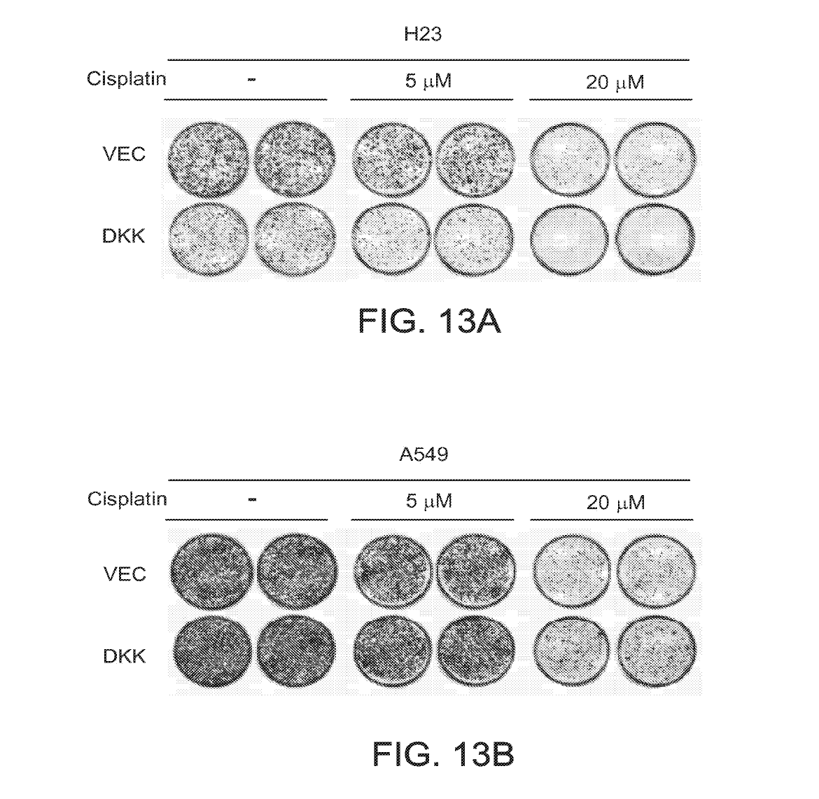

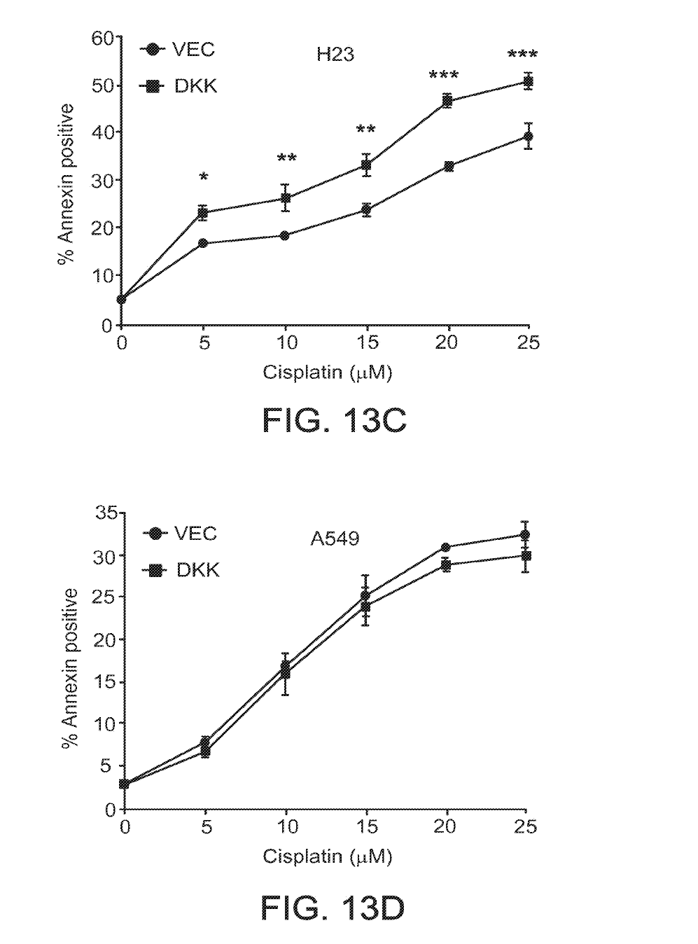

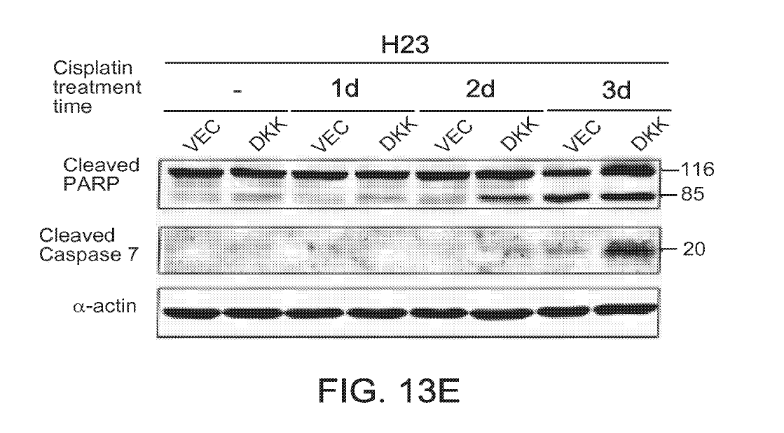

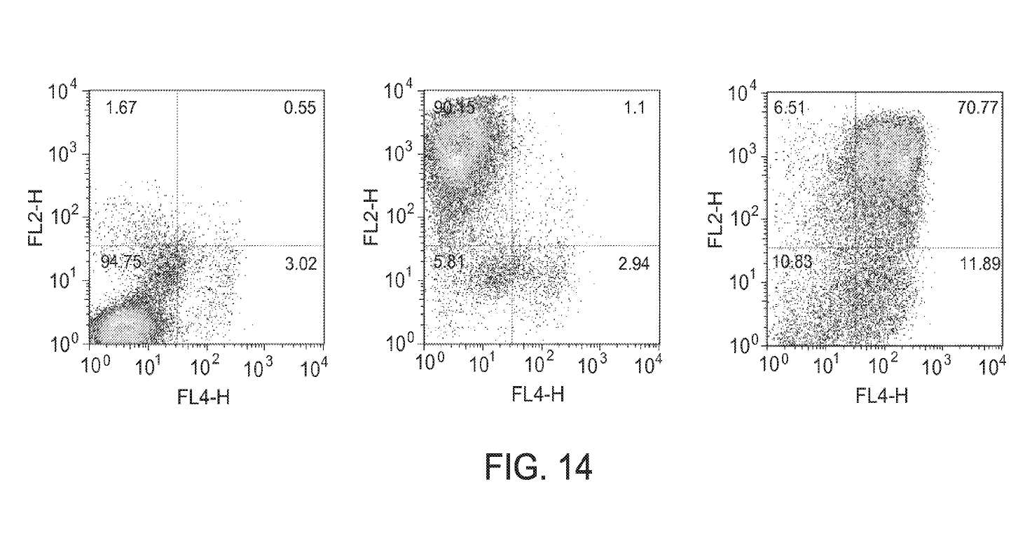

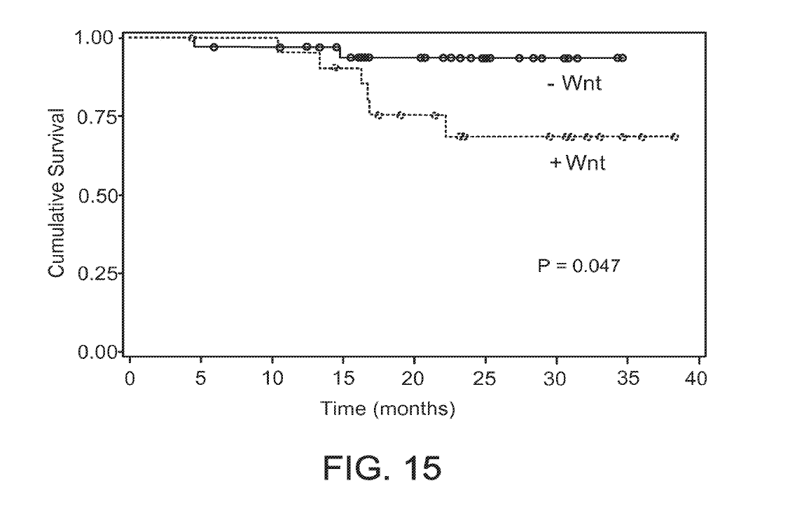

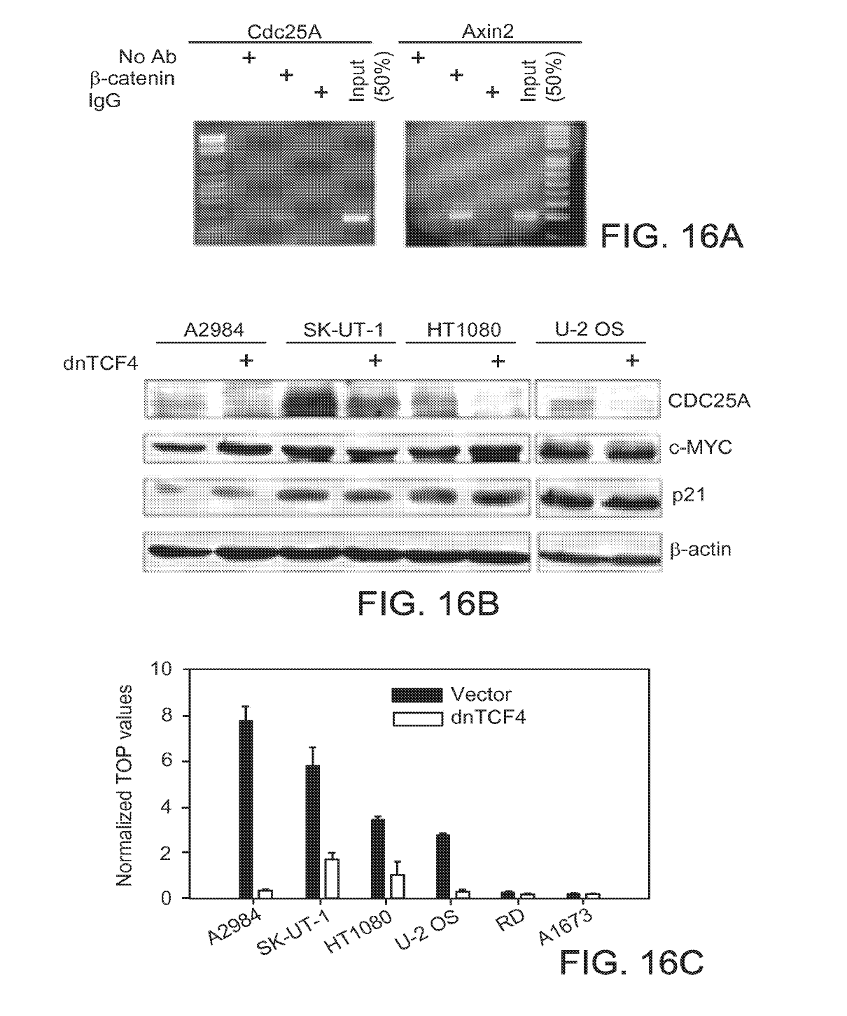

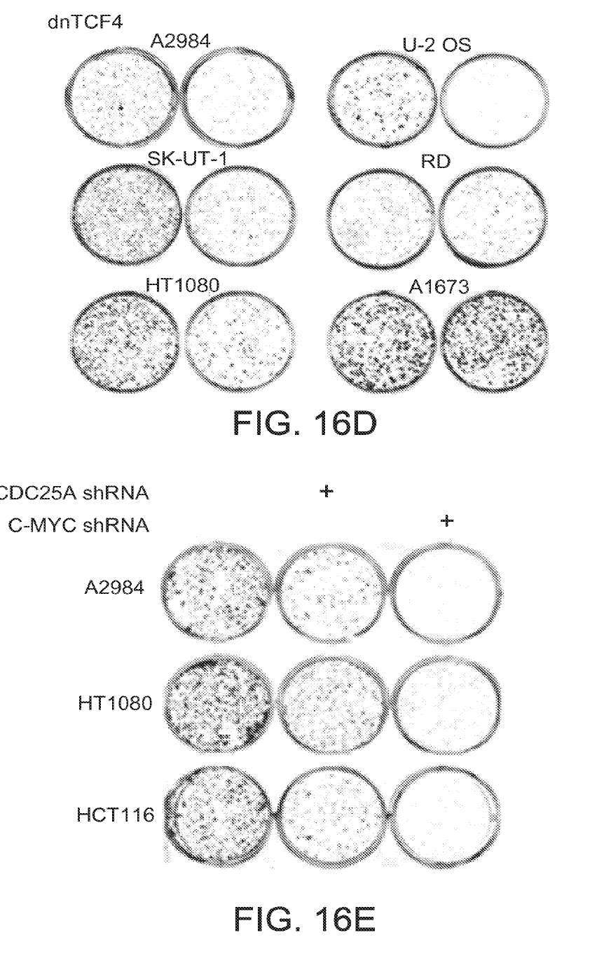

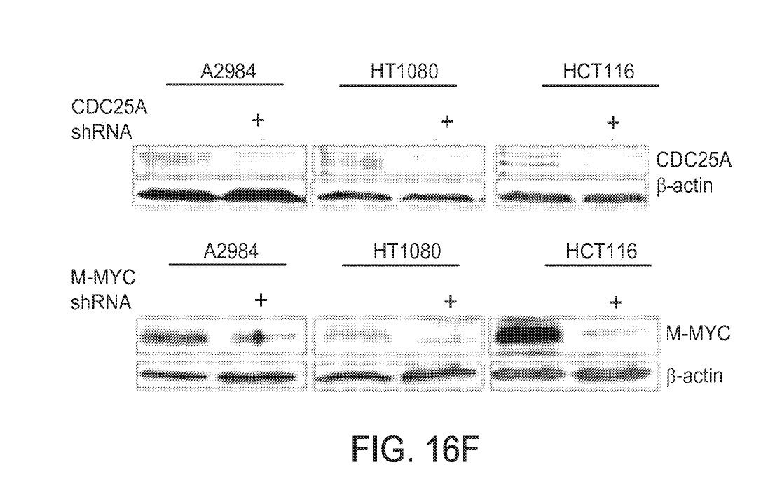

Another aspect of this invention is the identification of Wnt autocrine pathway activation in primary human tumors that had not been disclosed to exhibit lesions in this pathway including astrocytomas, glioblastomas, osteogenic and other sarcomas. Wnt pathway activation in these tumor types was also established using tumor cell lines. In the tumor lines, other methods can be applied to detection of pathway activation to confirm pathway activation. FIGS. 9-11 demonstrate the activation of canonical Wnt signaling in human astrocytoma cell lines, human sarcoma cell lines, and human osteosarcoma cell lines, respectively. The level of β-catenin in these cell lines was determined by lysing the cells in culture and doing the GST-pulldown method without subjecting cells to freezing and thawing and potentially releasing β-catenin from its bound form to cadherins. These findings further establish the importance of the present diagnostic methods.

Another aspect of this invention is to test human tumor samples arising in a specific tissue by quantitative RT-PCR or expression RNA profiling and demonstrate expression of genes that are upregulated in stem/progenitor cells or repressed compared to differentiated cells of the same tissue. Axin2 represents an example of a Wnt target gene that appears to universally upregulated in Wnt activated tumors independent of their tissue origin is Axin2. Thus, Axin2 expression at high level in a tumor is a particularly strong indication of Wnt pathway activation. Myc and DKK2 reflect Wnt responsive genes that may be upregulated in Wnt canonical activated tumors depending on tissue type.

List of target genes of Wnt signaling in various tumor types

| | | |
|---|---|---|
| Gene/s | Affected by Wnt | System |
| Axin2 | T | Universal |
| c-myc | T | Tissue-specific |
| DKK2 | T | Tissue-specific |
| Differentiation-associated | 1, | Tissue-specific |
| +Alkaline phosphatase, | | |
| Glial Fibrillary Acidic Protein | | |
| (GFAP), MUC-1, etc.+ | | |
| Stem/Progenitor-associated | T | |
| +Oct-4, Nanog, Nestin, etc.+ | Tissue-specific | |

Immunostaining for proteins whose genes such as Axin2 are upregulated in Wnt activated tumor samples is another approach to identify Wnt pathway involvement in such tumors.

Another aspect of this invention is the demonstration that therapeutic methods that inhibit activated Wnt pathway can have a profound cell killing and/or cytostatic effect on such tumor cells in the absence of other therapeutic modalities, and can cooperate with other therapeutic modalities to enhance tumor cell killing. See, for example, FIG. 13, which demonstrates that DKK1 specifically sensitizes autocrine Wnt NSCLC cells to cisplatin treatment. These findings disclose a strong rationale for therapeutic modalities that target this signaling pathway for use alone or in combination with standard chemo/irradiation therapies as well as evidence for the specific therapeutic efficacy of Wnt signaling downregulation in those tumors, which show

pathway activation by the approaches disclosed in this invention. The importance of a robust method to detect Wnt pathway activation in tumor samples derives from the present evidence that Wnt activated tumor cells can be hypersensitive to cell death induced by pathway downregulation alone or in combination with standard cancer therapies.

Additionally, knowledge of tumor phenotype is critical in personalizing therapy as exemplified by HER2 positive tumors, which specifically respond to herceptin while breast tumors lacking this amplified gene are unresponsive. Similarly, the results of this invention show complementation with chemotherapy in lung tumor cells specifically downregulated for Wnt signaling whereas the same DNTCF has no effect on therapy of a Wnt negative lung tumor cell.

### Materials and Methods

### Cell Culture

Human NSCLC cell lines Al 146, A549, A427 were grown in DMEM medium (Invitrogen) supplemented with 10% FBS (Invitrogen). NSCLC lines H23, H1819, H1355, H2347, HCC193, HCC515, H358, Hl 171, HCC46I, HCC827, H1299, HCC15, H460 and SCLC cell lines H 128, H82, H209, H2081, Hl 184, H889 and H249 (all cell lines were obtained from ATCC with the exception of Al 146, which was established using the method described in Giard et al., J. Natl. Cancer Inst., 1973, 51:1417-1423) were grown in RPMI medium (Invitrogen) supplemented with 10% FBS (Invitrogen). Immortalized human bronchial epithelial cell line, NL20 (Schiller et al, 1992) was purchased from ATCC (American Type Culture Collection) and grown in a specific growth medium as recommended. The normal Human Bronchial Epithelial cells (NHBE) were purchased from Lonza (Allendale, N.J., USA) and cultured in the recommended medium (Lonza). AU cells were cultured at 37° C. in 5% CO₂.

### Analysis of Uncomplexed β-Catenin and Immunoblot Analysis

GST-E-cadherin binding and immunoblot analysis was performed as previously described (Bafico et al, 1998), with the exception that for human tumor samples it was done with freshly frozen or stored tissue under cold, mild-detergent conditions. Specifically, the following protocol to assay for uncomplexed (active) β-catenin in human tumor samples was used:

Tumor and normal tissue samples are fresh frozen in liquid nitrogen (snap frozen) and stored at −8O° C. or liquid nitrogen. If stored in OCT, frozen sections can be used for this analysis, so that homogenization of tissue can be performed without losses of protein making the assay highly quantitative and reproducible.
GST beads incubated with E-cadherin lysate (as described by Bafico, 2004) Small Mortar: 3 cm in diameter Small Pestle: 1 cm at the base
10-15 small layers from a frozen tissue are scraped off and deposited in center of chilled mortar
The layers are ground thoroughly with a pestle (pre chilled)
30O uL of lysis buffer are added and followed by further grinding
The lysate, a cloudy yellowish or reddish liquid with some visible pieces of tissue, is transferred to a to clean (1.5 mL) centrifuge tube
Another 30O uL of lysis buffer is used to rinse the mortar and pestle accompanied by further grinding and added to the collect in the aforementioned centrifuge tube
Samples are kept on ice for 15-20 minutes with continuous and vortexing throughout
A centrifugation for 15 minutes at 14,000 RPM and 4° C. will produce a visible pellet and a somewhat clear supernatant
The supernatant is transferred to fresh tube
Protein concentration is measured following the Bradford Assay using a 1:500 dilution-4 uL in I mL total volume
100-200 uL of lysis buffer is added to samples with protein concentration higher than 2 ug/uL in order to dilute the concentration to anywhere between 1-2 ug/uL
Protein concentration is measured once more
The following amounts are used:
Pull down: 500 ug
Lysate for running: 20 ug
20O uL of E-cadherin complexed GST beads is added for pull down followed by a 1 hour incubation at 4° C. with rotation
Washing of beads is as follows:
1. Spin: 5 min, 14K RPM, 4° C.
2. Aspirate supernatant
3. Add I mL lysis buffer
Wash is repeated three times. The last wash is followed by the addition of SDS loading dye
Samples are run in an 8% SDS-polyacrylamide gel and Western blotting is carried out as usual Amount of tissue utilized for the uncomplexed β-catenin assay-equivalent of 15-20 frozen sections

| Lysis Buffer Recipe | |
|---|---|
| Tris pH7.4 | 50mM |
| Sodium Chloride (Fisher S271-1) | 190mM |
| Igepal CA-630 (Sigma 18896-5OmL) | 1% |
| added fresh before lysing: | |
| Aproptinin | 1 Oug/mL |
| PMSF (Gibco BRL 15521-016) | 2mM |
| Sodium Vanadate (Fisher S454-50) | 2mM |
| Sodium Fluoride (Sigma S 1504-100G)1mM | |

Concentration of serum free conditioned medium obtained from vector or DKK1 expressing cells was performed using Amicon Ultra-15 centrifugal filters (Millipore, Ireland). Expression of FRP1-HA was detected in cell lysates using anti HA antibody. For immunoblot analysis the following primary antibodies were used: HA, Flag and c-Myc (9E10) (Hybridoma Center, Mount Sinai School of Medicine, New York), β-catenin and p21 (BD Pharmingen), PARP and cyclin D1 (Santa Cruz Biotechnology), caspase 7 (Cell signaling). Anti-mouse IgG or anti-rabbit IgG secondary antibodies conjugated to Horseradish Peroxidase or Alexa Fluor 680 were purchased from Amersham Bioscience (GE Healthcare, UK) or from Molecular Probes (Oregon, USA), respectively. Quantification of signal immunoreactivity was obtained using enhanced chemiluminescence detection system (Amersham, N.J., USA) or the Licor Odyssey Imaging system (LI-COR).

### FACS Analysis of TCF Mediated GFP Reporter Activity

Cells infected with TOP or FOP EGFP reporter lentiviruses were transferred to polystyrene tubes (Falcon, N.J., USA) and subjected to FACS analysis (Becton Dickinson FACScan, NJ, USA) using Cell Quest 3.2 software (Becton Dickinson).

### Quantification of TCF Mediated Luciferase Reporter Activity

24 hours before infection, stable reporter cell lines expressing TOP or FOP TCF luciferase and renilla luciferase were plated in 6 well plates at I×IO⁵ cells/well. The following day cells were infected with lentiviruses expressing different Wnt antagonists, selected with 2 μg/ml puromycin for 3 days, lysed and processed for luciferase reporter assay using the dual luciferase reporter Kit (Promega) according to the manufacturer's protocol. Luciferase reporter activity was calculated by dividing the ratio TOP/RL by the FOP/RL ratio. Results were normalized to the results with vector transduced cultures.

### FACS Analysis of Cell Cycle and Annexin-PI

For DNA content analysis, cells were trypsinized, combined with floating cells, washed with PBS, stained with propidium iodide (PI), using the CycleTEST Plus DNA reagent kit (Becton Dickinson) following the manufacturer instructions, and subjected to FACS analysis. For Annexin V-PI cells were treated as for cell cycle analysis, stained with Annexin-PI using the Annexin V-FITC apoptosis detection kit (R&D systems) according to the manufacturer instructions, and subjected to FACS analysis. Results were analyzed using Cell Quest 3.2 software (Becton Dickinson).

### Cell Growth Analysis Assay

Transduced and marker selected cells were trypsinized, counted and 1-2×10⁴ cells plated into 60 mm tissue culture dishes. At 2-3 weeks, cells were washed with PBS, fixed in 10% methanol/acetic acid solution and stained with 1% crystal violet.

### Statistical Analysis

Statistical analysis was performed using two-way analysis of variance (ANOVA) with Bonferroni multiple testing corrections employing the Prism 5 software (GraphPad Prism software, San Diego, Calif.). A p value <0.05 was considered statistically significant. Values are represented as arithmetical mean±SD.

### Lentiviral Constructs

TCF reporter lentiviral constructs driving the expression of EGFP were generated by cloning a PCR amplified cassette containing seven wild-type or mutated TCF/LEF binding sites with a minimal TATA promoter from Super TOP/FOP flash between CIaI and BamHI sites of pRRL-SIN-cPPT-PGK-GFP, replacing the PGK promoter. TCF reporter lentiviral constructs driving the expression of firefly hiciferase were generated by replacing EGFP in the TOP or FOP TCF-EGFP lentiviral constructs with PCR amplified firefly luciferase. Renilla luciferase (RL) lentiviral construct driven by a constitutive PGK promoter, used to normalize for infection efficiency, was generated by cloning a PCR amplified RL instead of EGFP in pRRL-SIN-cPPT-PGK-GFP. Lentiviral vectors used for constitutive or inducible expression were generated as follows: NSPI-CMV-MCS-myc-His lentiviral expression vector was constructed by inserting a linker containing the restriction enzymes Nsil-Xbal-BstBI-MluI-Clal and Sail between CIaI and Sail sites of pRRL-SIN-cPPT-PGK-GFP lentiviral vector. A cassette containing SV40 promoter driving Puromycin selection marker was digested from pBabe-puro using Accl and CIaI and cloned into BstBI site. PGK-GFP cassette was then inserted into the CIaI and Sail sites to generate NSPI-PGK-GFP. CMV promoter with multiple cloning sites (MCS) was digested from pCDAN3.1+ Neo (Invitogen) using MIuI and Xhol replacing the PGK-GFP to generate NSPI-CMV-MCS. Lastly, the CMV promoter was replaced with CMV

promoter containing MCS and myc-His cassette from pCDNA3.1-myc-His (Invitrogen) using 1 MIuI and Pmel to generate NSPI-CMV-MCS-myc-His. To generate an inducible lentiviral expression vector the PGK promoter was replaced in NSPI-PGK-GFP with a tetracycline response element (TRE) containing minimal CMV promoter cassette generating NSPI-TRE-GFP Lentiviral vector expressing the tetracycline trans-activator (tTa) under a constitutive CMV promoter was generated by cloning tTa fragment digested with EcoRT and BamHI from pRev-Tet-Off-IN (Clontech) into pCDNA3.1 (Invitrogen). CMV-tTA cassette was then digested from pCDNA3.1-tTA with MIuI and Xhol and cloned between MIuI and Sail sites in NSBI-PGK-MCS lentiviral vector containing blasticidin selection. Flag-tagged DKK1, HA-tagged FRP1, DNTCF4 or DNTCF4-mOrange were all cloned between BamHI and Xhol of NSPI-CMV-MCS-myc-His or NSPI-TRE-GFP to generate the corresponding constitutive or inducible lentiviral vectors.

### Production of Lentiviruses

Second-generation VSV-G pseudotyped high titers lentiviruses were generated by transient co-transfection of 293T cells with a three-plasmid combination as follows: One T75 flask containing I×IO⁷ 293 T cells was transfected using FuGENE 6 (Roche) with 5 μg lentiviral vector, 3.75 μg pCMV Δ8.91 and 1.25 μg pMD VSV-G. Supernatants were collected every 12 hr between 36 to 96 hr after transfection, pulled together and frozen at −7O° C.

### Lentiviral Transduction

For lentiviral transduction, I×IO⁵ cells/well were seeded in 6 well tissue culture plates and infected the following day with TOP or FOP EGFP lentiviruses. When cultures reached confluency, cells were trypsinized and processed for FACS analysis. For TCF luciferase reporter

analysis, cells were co-infected with TOP or FOP firefly luciferase mixed with a lentivirus expressing renilla luciferase (RL) used to normalize for infection efficiency (1:20-1:40 ratio). To assess the effects of FRP1 and DKK1 on TCF luciferase reporter activity, stable reporter cell lines were plated in 6 well plates and infected with vector, FRP1 or DKK1 lentiviruses. Cells were selected for 3 days with puromycin, lysed and processed for dual luciferase analysis. To generate stable inducible lines, cells were infected consecutively with tetracycline trans-activator (tTa) expressing lentivirus, selected for two weeks with 5-10 μg/ml blasticidin, followed by a second infection with lentiviruses expressing inducible FRP1, DKK1, DNTCF-4 or DNTCF-4-mOrange or empty vector, and selected for 4-7 days in 2 μg/ml puromycin in the presence of 10 ng/ml doxycycline. For induction of the antagonists, cells were trypsinized, washed with PBS and plated into 10 cm plates in the presence or absence of doxycyclin. Control cells expressing tTa or empty vector were processed in the same way. All infections were performed for 16 hr in the presence of 8 μg/ml polybrene.

### ShRNA Knockdown of Wnt2 and Wnt3a Expression

An shRNA construct targeting human Wnt2 was obtained from Open Biosystems. The 21 bp sequence was 5′-GCTCATGTACTCTCAGGACAT-3′ (SEQ ID NO: 1). An shRNA construct targeting GFP containing 21 bp sequence 5′ GCTCATGTACTCTCAGGACAT-3′ (SEQ ID NO: 2), was obtained from Addgene. An shRNA construct targeting human Wnt3a was generated and had the following sequence 5′-GGCGTGGCTTCTGCAGAA-3′ (SEQ ID NO: 61). Viruses were produced in 293T cells using FuGENE 6 (Roche) as described above.

### RT-PCR and Quantitative Real Time PCR

Total RNA was isolated from cells using the Trizol Reagent (Invitrogen) according to the manufacturer's protocol. Semi-quantitative RT-PCR screen was performed using One Step RT-PCR Kit (Invitrogen) according to the manufacturer's protocol. PCR products were separated on 1% agarose gel. 5 μg total RNA was reverse transcribed using Superscript II reverse transcriptase (Invitrogen). Quantitative PCR was performed using SybrGreen 2× master mix (Roche) on a MJ Opticon or Stratagene MxPro3005. 50 ng cDNA were amplified as follows: 94° C. for 15 min, 94° C. for 15 s, 60° C. for 30 s, 72° C. for 1 min. Steps 2 through 4 were repeated for 40 cycles. Each reaction was performed in duplicate, and results of 3 independent experiments were used for statistical analysis. Relative mRNA expression levels were quantified using the ΔΔC(t) method (Pfaffl, 2001). Results were normalized to those for TATA Binding Protein (TBP). Primer sequences can be found in Tables 4 and 5.

### Sequencing of CTNNB1 Exon 3

Genomic DNA extracted from NSCLC cell lines using the DNeasy extraction kit (Qiagene, Maryland), was PCR amplified using primers flanking β-catenin (CTNNB1) exon 3 (forward 5′-TTGATGGAGTTGGACATG [SEQ ID NO: 3]; reverse 5′-CAGCTACTTGTTCTTGAG [SEQ ID NO: 4]). Gel purified PCR fragments were sequenced at the DNA sequencing core facility of Mount Sinai Medical Center, New York.

### Tissue Specimens

Human lung adenocarcinomas and adjacent normal lung tissue were randomly selected from anonymized bank. All tumors were confirmed as NSCLC by pathological examination. Tissues were preserved by immersing in OCT and snap frozen. Cryosections were stored in −70° C. until processed.

Table 3-Wnt signaling activation in human lung cancer lines

| | | | |
|---|---|---|---|
| Cell line (NSCLC) | Uncompleted | I3?+0catenin | TCF-GFP reporter activity |
| level | (TOP/FOP) | | |
| A549 | -1+30 | - | |
| 11460 | - | - | |
| 111299 | - | - | |
| 111171 | - | - | |
| HCC193 | -1+30 | - | |
| 1123 | +30+30 | +30+30 | |
| 111819 | +30+30+30 | +30+30 | |
| A1146 | +30+30+30 | +30+30 | |
| 111355 | +30+30/+30+30+30 | +30/+30+30 | |
| 112347 | +30+30 | +30/+30+30 | |
| 11358 | +30+30 | +30+30 | |
| HCC515 | +30+30+30 | +30/+30+30 | |
| A427 | +30+30+30+30 | +30+30+30 | |
| HCC15 | +30+30+30 | +30+30+30+30 | |
| 11461 | -1+30 | - | |
| HCC827 | -1+30 | - | |
| 11128 | - | NK | |
| 1182 | - | NC | |
| 11209 | - | ND | |
| 112081 | - | NC | |
| 111184 | - | ND | |
| 11889 | - | ND | |
| 11249 | - | ND | |
| * ND - Not Determined | | | |
| Table 3. 16 Human NSCLC cell lines and 7 SCLC cell lines (all obtained from ATCC with the | | | |
| exception of Al 146, which was established using the method described in Giard et al., J. Natl. | | | |
| Cancer Inst., 1973, 51 : 1417-1423) were analyzed for expression of uncomplexed f3-catenin and | | | |
| TOP and FOP TCF-GFP reporter activity as described in methods. Relative levels of uncomplexed | | | |
| f3-catenin were approximated based on comparison between different lines analyzed at the same | | | |
| time. (ND ? Not Determined). | | | |

TABLE 4
RT-PCR primers for human Wnt family members

| | | | | | | Product |
|---|---|---|---|---|---|---|
| | Gene | Primer Fwd Sequence | SEQ | Primer Rev Sequence | | Size |
| Gene | ID | 5′->3′ | ID | 5′->3′ | SEQ | (bp) |
| WNT1 | 7471 | GTGGGGTATTGTGAACGTAG | 5 | GGTTGCCGTACAGGACGC | 6 | 680 |
| WNT2 | 7472 | GCGCCAAGGACAGCAAAG | 7 | GCGGTTGTCCAGTCAGCGTTC | 8 | 646 |
| WNT2B | 7482 | CCGACACCATGACCAGCG | 9 | TCCAGGCACTCTGCCTTC | 10 | 645 |
| WNT3 | 7473 | CTCGGTGGCACCAGGGTC | 11 | CTTCCCATGAGACTTCGCTG | 12 | 995 |
| WNT3A | 89780 | GAAGCAGGCTCTGGGGAG | 13 | GGAGTACTGCCCCGTTTAGG | 14 | 1119 |
| WNT4 | 54361 | GAGGAGGAGACGTGCGAG | 15 | GCGTGGCTCCACCTCAGT | 16 | 627 |
| WNT5A | 7474 | GTCTTCCAAGTTCTTCCTAGTG | 17 | CTTGCCCCGGCTGTTGAG | 18 | 787 |
| WNT5B | 81029 | TGGGCTCAGCTTCTGACAGAC | 19 | CTCCAGCCGGCCCTTGCG | 20 | 784 |
| WNT6 | 7475 | CACGTCGGCGGACTGTGG | 21 | CTTGCCGTCGTTGGTGCC | 22 | 741 |
| WNT7A | 7476 | CGCTGCCTGGGCCACCTC | 23 | CTCGTCCCGGTGGTACTG | 24 | 416 |
| WNT7B | 7477 | CGCAAGTGGATTTTCTACGTG | 25 | GAAGGTGGGCTGCCGCAG | 26 | 738 |
| WNT8A | 7478 | AACCTGTTTATGCTCTGGGC | 27 | CTCTCAGCTGCCGCTTATCC | 28 | 673 |
| WNT8B | 7479 | CTTTTCACCTGTGTCCTCCAAC | 29 | CCGGGTAGAGATGGAGCG | 30 | 699 |
| WNT9A | 7483 | GCGGCCTTCGGGCTGACG | 31 | GGAGAAGCGGCCAGCCAG | 32 | 771 |
| WNT9B | 7484 | AGGATTGGGCACTGCGGC | 33 | GTGAGTACTTGCTGGGCCG | 34 | 782 |
| WNT10A | 80326 | ACAAGATCCCCTATGAGAGTC | 35 | GGGCAGGGCTGGGTGTTC | 36 | 257 |
| WNT10B | 7480 | CCTCGGGCCTCGCGGGTC | 37 | GCCCTCAGCCGATCCTGC | 38 | 445 |
| WMT11 | 7481 | ATATCCGGCCTGTGAAGGAC | 39 | CAAGTGAAGGCAAAGCACAA | 40 | 424 |
| WMT16 | 51384 | TCACCACTTGCCTCAGGG | 41 | GTTTTCTTTGCCCGTGGTGTTTC | 42 | 548 |
| GAPDH | 2597 | GGAAGGTGAAGGTCGGAGTC | 43 | GTGATGGCATGGACTGTGG | 44 | 541 |

*A11 primers span at least 1 intron

TABLE 5
Primers for Quantitative real time PCR

| | | Primer Fwd | | Primer Rev | | Product |
|---|---|---|---|---|---|---|
| | Gene | Sequence | SEQ | Sequence | | Size |
| Gene | ID | 5′->3′ | ID | 5′->3′ | SEQ | (bp) |
| WNT2 | 7472 | ACTCTCCAGGA | 45 | GAGGTCATTTT | 46 | 160 |
| | | CATGCTGGCT | | TCGTTGGCTT | | |
| WNT3a | 89780 | GCCCCACTCGG | 47 | GGGCATGATCT | 48 | 189 |
| | | ATACTTCT | | CCACGTAGT | | |
| AXIN2 | 8313 | ACTGCCCACAC | 49 | CTGGCTATGTC | 50 | 127 |
| | | GATAAGGAG | | TTTGGACCA | | |
| AIAT | 5265 | GAATCGACAAT | 51 | TGGGATGTATC | 52 | 125 |
| | | GCCGTCTTCT | | TGTCTTCTGGG | | |
| MUC1 | 4582 | AAGCAGCCTCT | 53 | GGTACTCGCTC | 54 | 248 |
| | | CGATATAACCT | | ATAGGATGGT | | |
| ICAMI | 3383 | GCCAACCAATG | 55 | AGGGTAAGGTT | 56 | 136 |
| | | TGCTATTCA | | CTTGCCCAC | | |
| CCSP | 7356 | TTCAGCGTGTC | 57 | ACAGTGAGCTT | 58 | 189 bp |
| | | ATCGAAACCC | | TGGGCTATTTT | | |
| | | | | T | | |
| TBP | 129685 | ATCAGTGCCGT | 59 | TTCGGAGAGTT | 60 | 150 |
| | | GGTTCGT | | CTGGGATTG | | |

*Primers span at least 1 Intron

### Example 2

### Analysis of Human Tumor Samples for Wπt Activation

Frozen sections of tumor samples were washed twice in PBS. Equivalent aliquots of 300 μg total cell lysates were subjected to precipitation with a GST-E cadherin fusion protein (as described in Bafico, A. et al, 2004). Total cell lysates (10 μg) and GST-E-cadherin precipitates were analyzed by immunoblot using a mAb antibody against β-catenin (BD Pharmingen, San Jose, Calif.). A summary of human tumor samples analyzed for Wnt activation by this method is provided in the table below:

| | |
|---|---|
| Tumor | Wnt Activated / Total Analyzed |
| Breast | 2/7 |
| Ovarian | 4/7 |
| Lung | 22/57 |
| Sarcoma* | 13/29 |
| * Includes high proportion of Wnt negative liposarcomas. | |

### Example 3

### Inhibition of Activated Autocrine Wnt Signaling in HA235 GIioblasoma Cells Inhibits their Proliferation and Induces Apoptosis

8 out of 17 tested brain tumor cell lines (astrocytoma/glioblastoma) were positive in tests for Wnt activation as determined assays for uncomplexed β-catenin: A235, A382, HA153A, HA153B, HA690, HA197, A826, and A597.

Annexin positivity (which detects loss of plasma membrane, one of the earliest features of apoptosis) was determined by flow cytometry analysis using Annexin V conjugated to APC following DNTCF expression in a Wnt positive brain tumor line HA235 glioblastoma. As shown in FIG. 14, dominant negative TCF4-mOrange (DN-mO), an inhibitor of autocrine Wnt signaling (Akiri G. et al., Oncogene 2009), induces apoptosis in HA235 glioblastoma cells as evident by Annexin V staining.

### Example 4

### Wnt Pathway Activation Predicts Increased Risk of Tumor Recurrence in Patients with Stage I Non-Small Cell Lung Cancer

57 patients treated with surgical resection for stage I NSCLC between June 2006 and May 2008 were selected from a database linked to the cancer tissue biorepository containing fresh frozen tumor as well as a normal lung tissue specimens linked to each patient. A glutathione-S-transferase (GST) pull-down assay combined with immunoblot analysis was used to assess the levels of uncomplexed and total β-catenin in tissues. The β-catenin gene was

tested for oncogenic mutations in tumors with activated Wnt signaling, and cancer recurrence rates were compared in Wnt pathway positive and negative tumors.

38.6% (n=22) of tumors were scored as Wnt positive with only one exhibiting a β-catenin oncogenic mutation. Thus, the great majority of Wnt activated primary tumors, as with NSCLC tumor lines, likely exhibit Wnt autocrine activation. Patients with Wnt positive tumors experienced a significantly higher rate of cancer recurrence than those whose tumors were Wnt negative (27.3%, n=6 vs. 5.7%, n=2) (FIG. 15). Moreover, there were 5 patients with distal tumor recurrence in the Wnt positive group compared to 1 in the other group (22.7% vs. 2.9%, p=0.036).

The present study establishes a role for Wnt pathway activation in a substantial fraction of primary human NSCLCs. Moreover, increased levels of Wnt pathway activation were associated with a higher rate of cancer recurrence in patients with Stage I NSCLC. These findings suggest that Wnt activation reflects a more aggressive tumor phenotype and identifies patients who may benefit from more aggressive therapy in addition to resection.

### Example 5

### Downregulation of CDC25A, a Novel Wnt Target Gene, Inhibits Proliferation of Human Sarcoma Cells In Vitro

In sarcomas, CDC25a is a Wnt target gene as defined by CHIP analysis with β-catenin at the CDC25a promoter. The presence of β-catenin at the CDC25a promoter indicates that β-catenin may recruit other factors, including TCF/LEF 1 transcription factors, to induce the transcription of CDC25A. To determine whether CDC25A is a direct target of Wnt signaling in sarcoma cells, chromatin immunoprecipitation was conducted on DNA extracted from U-2 OS, a Wnt autocrine sarcoma cell line (ATCC) (FIG. 16A). Monoclonal antibody against β-catenin (BD Biosciences) was used in immunoprecipitation. Primers to amplify Axin 2 were used as

positive control. As shown in FIG. 16B, downregulation of Wnt signaling in sarcoma cells A2984, SK-UT-I, HT1080, and U-2 OS using dominant-negative TCF4 (dnTCF4) results in simultaneous decrease in CDC25A expression.

Downregulation of Wnt signaling in sarcoma cells expressing dnTCF4. A2984, SK-UT-I, HT 1080, U-2 OS, RD, and A1673 sarcoma cells stably expressing TOP luciferase and a normalizer, renilla luciferase, were used in this assay. FIG. 16D demonstrates that dnTCF4 expression induces growth arrest in sarcoma cells. Human sarcoma cells A2984 (developed from a primary tumor using the method described in Giard et al., J. Natl. Cancer Inst., 1973, 51:1417-1423), SK-UT-I (ATCC), HT1080 (ATCC), U-2 OS (ATCC), RD (ATCC), and A1673 (developed from a primary tumor using the method described in Giard et al., J. Natl. Cancer Inst, 1973, 51:1417-1423) were stably infected (lentiviral transduced) with dnTCF4 or an empty vector control and selected in puromycin for 3 days. Cells were plated at 1000 cells/60 mm density and cultured for 10 days. Cells were fixed using formaldehyde and stained with crystal violet. As shown in FIG. 16D, dnTCF4 expression did not affect proliferation in a Wnt signaling negative cell line, A 1673, or a low Wnt positive sarcoma cell line, RD, while, the expression of dnTCF4 in A2984, SK-UT-I, HT1080 and U-2 OS cells inhibited proliferation in vitro. FIG. 16E shows that knockdown of CDC25A or c-myc induces growth arrest in sarcoma cells. HCTl 16 (ATCC), a human colon cancer cell line was used for comparison. A2984, HT1080 and HCTl 16 cells were stably infected (lentiviral transduced) with either empty vector control or shRNA specific for CDC25A (5′CCAGGGAATTTCATTCCTC3′; SEQ ID NO: 62) or c-myc (5′GATGAGGAAGAAATCGATGS′; SEQ ID NO: 63) and selected in puromycin and plated at 1000 cells/60 mm plate and cultured for 10 days. Cells were fixed and stained with crystal violet. FIG. 16F shows Western blotting demonstrating specific downregulation of CDC25A or c-myc after shRNA expression in A2984, HT1080 and HCTl 16

cells. Both CDC25A and c-MYC shRNAs inhibited proliferation to varying degrees in all three cell lines tested, implying the critical importance of each of these genes to the proliferation of these cells.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

### REFERENCES

Akiri G, Cherian M M, Vijayakumar S, Liu G, Bafico A, Aaronson S A (2009). Wnt pathway aberrations including autocrine Wnt activation occur at high frequency in human non-small-cell lung carcinoma. Oncogene. 2009 May 28; 28(21):2163-72.
Bafico A, Gazit A, Wu-Morgan S S, Yaniv A, Aaronson S A (1998). Characterization of Wnt-1 and Wnt-2 induced growth alterations and signaling pathways in NIH3T3 fibroblasts. Oncogene 16: 2819-25.
Bafico A, Liu G, Goldin L, Harris V, Aaronson S A (2004). An autocrine mechanism for constitutive Wnt pathway activation in human cancer cells. Cancer Cell 6: 497-506.
Bemasconi N L, Wormhoudt T A M, Laird-Offringa I A (2000). Post-Transcriptional Deregulation of myc Genes in Lung Cancer Cell Lines. Am. J. Respir. Cell MoI. Biol. 23: 560-565.
Braga V M, Pemberton L F, Duhig T, Gendler S J (1992). Spatial and temporal expression of an epithelial mucin, Muc-1, during mouse development. Development 115: 427-437.
Clevers H (2006). Wnt/β-Catenin Signaling in Development and Disease. Cell 127: 469-480.
Ding L, Getz G, Wheeler D A, Mardis E R, McLellan M D, Cibulskis K et al (2008). Somatic mutations affect key pathways in lung adenocarcinoma. Nature 455: 1069-1075.
Giard D J, Aaronson S A, Todaro G J, Amstein P, Kersey J H, Dosik H et al (1973). In vitro cultivation of human tumors: establishment of cell lines derived from a series of solid tumors. J Natl Cancer Inst 51: 1417-23.
Guzman J, Izumi T, Nagai S, Costabel U (1994). ICAM-I and integrin expression on isolated human alveolar type II pneumocytes. Eur Respir J 7: 736-739.
He B, You L, Uematsu K, Xu Z, Lee A Y, Matsangou M et al (2004). A monoclonal antibody against Wnt-1 induces apoptosis in human cancer cells. Neoplasia 6: 7-14.
Jho E H, Zhang T, Domon C, Joo C K, Freund J N, Costantini F (2002). Wnt/β-catenin/Tcf signaling induces the transcription of Axin2, a negative regulator of the signaling pathway. MoI Cell Biol 22: 1172-83.
Kawano Y, Kypta R (2003). Secreted antagonists of the Wnt signalling pathway. J Cell Sci 116: 2627-34.
Kim C F B, Jackson E L, Woolfenden A E, Lawrence S, Babar I, Vogel S et al (2005). Identification of Bronchioalveolar Stem Cells in Normal Lung and Lung Cancer. Cell 121: 823-835.
Klaus A, Birchmeier W (2008). Wnt signalling and its impact on development and cancer. Nat Rev Cancer 8: 387-398.
Minna J D, Roth J A, Gazdar A F (2002). Focus on lung cancer. Cancer Cell 1: 49-52.
Morin P J, Sparks A B, Korinek V, Barker N, Clevers H, Vogelstein B et al (1997). Activation of β-Catenin-Tcf Signaling in Colon Cancer by Mutations in β-Catenin or APC. Science 275: 1787-1790.
Mucenski M L, Nation J M, Thitoff A R, Besnard V, Xu Y, Wert S E et al (2005). β-Catenin regulates differentiation of respiratory epithelial cells in vivo. Am J Physiol Lung Cell MoI Physiol 289; L971-979,
Nakamura N, Kobayashi K, Nakamoto M, Kohno T, Sasaki H, Matsuno Y et al (2006). Identification of tumor markers and differentiation markers for molecular diagnosis of lung adenocarcinoma. Oncogene 25: 4245-55.
Ohgaki H, Rros J M, Okamoto Y, Gaspert A, Huang H, Kurrer M O (2004). APC mutations are infrequent but present in human lung cancer. Cancer Lett 207: 197-203.
Okubo T, Hogan B L (2004). Hyperactive Wnt signaling changes the developmental potential of embryonic lung endoderm. J Biol 3: 11.
Pfaffl M W (2001). A new mathematical model for relative quantification in real-time RT-PCR. Nucleic Acids Res 29: e45.
Polakis P (2007). The many ways of Wnt in cancer. Current Opinion in Genetics & Development 17: 45-51.
Reya T, Clevers H (2005). Wnt signalling in stem cells and cancer. Nature 434: 843-50.
Reynolds S D, Zemke A C, Giangreco A, Brockway B L, Teisanu R M, Drake J A et al (2008). Conditional Stabilization of β-Catenin Expands the Pool of Lung Stem Cells. Stem Cells 26: 1337-1346.
Richardson G E, Johnson B E (1993). The biology of lung cancer. Semin Oncol 20: 105-27.
Sansom O J, Meniel V S, Muncan V, Phesse T J, Wilkins J A, Reed I C R et al (2007). Myc deletion rescues Ape deficiency in the small intestine. Nature 446: 676-679.
Schiller J H, Bittner G, Oberley T D, Kao C, Harris C, Meisner L F (1992). Establishment and characterization of a SV40 T-antigen immortalized human bronchial epithelial cell line. In Vitro Cell Dev Biol Hk; 461-4.
Shigemitsu K, Sekido Y, Usami N, Mori S, Sato M, Horio Y et al (2001). Genetic alteration of the β-catenin gene (CTNNB1) in human lung cancer and malignant mesothelioma and identification of a new 3p21.3 homozygous deletion. Oncogene 20: 4249-57.
Sunaga N, Kohno T, Kolligs F T, Fearoit E R, Saito R, Yokota J (2001). Constitutive activation of the Wnt signaling pathway by CTNNB1 (β-catenin) mutations in a subset of human lung adenocarcinoma. Genes Chromosomes Cancer 30: 316-21.
van de Wetering M, Sancho E, Verweij C, de Lau W, Oving I, Hurlstone A et al (2002). The [(3]-Catenin/TCF-4 Complex Imposes a Crypt Progenitor Phenotype on Colorectal Cancer Cells. Cell 111: 241-250.
Veeman M T, Slusarski D C, Kaykas A, Louie S H, Moon R T (2003). Zebrafish Prickle, a Modulator of Noncanonical Wnt/Fz Signaling, Regulates Gastrulation Movements. Current Biology 13: 680-685.
Venembre P, Boutten A, Seta N, Dehoux M S, Crestani B, Aubier M et al (1994). Secretion of alpha 1-antitrypsin by alveolar epithelial cells. FEBS Lett 346: 171-4.
You L, He B, Xu Z, Uematsu K, Mazieres J, Mikami I et al (2004). Inhibition of Wnt-2-mediated signaling induces programmed cell death in non-small-cell lung cancer cells. Oncogene 23: 6170-4.
Zhang Y, Goss A M, Cohen E D, Kadzik R, Lepore J J, Muthukumaraswamy K et al (2008). A Gata6-Wnt pathway required for epithelial stem cell development and airway regeneration. Nat Genet 40: 862-870.

1. A method of determining whether a canonical Wnt signaling is activated in a tumor isolated from a subject comprising measuring the amount of uncomplexed β-catenin in the tumor.

2. The method of claim 1, wherein the tumor is derived from tissue which has been rapidly frozen after its isolation from the subject.

3. The method of claim 1, wherein the level of uncomplexed β-catenin is measured under mild detergent conditions.

4. The method of claim 3, wherein the mild detergent conditions comprise the use of a buffer that contains approximately 1% NP-40 or equivalent non-ion detergent.

5. The method of claim 1, wherein the uncomplexed β-catenin is captured using a soluble or immobilized E-cadherin protein or a fragment thereof containing β-catenin binding domain.

6. The method of claim 5, wherein the E-cadherin protein or a fragment thereof is fused to a tag.

7. The method of claim 6, wherein the tag is GST, His tag or FLAG.

8. A method of determining whether a canonical Wnt signaling is activated in a tumor comprising the steps of
(a) preparing a lysate of the frozen tumor tissue sample under mild-detergent conditions,
(b) incubating the lysate with soluble or immobilized E-cadherin protein or a fragment thereof containing β-catenin binding domain,
(c) isolating the resulting E-cadherin/β-catenin complex, and
(d) detecting the E-cadherin/β-catenin complex.

9. The method of claim 8, wherein step (d) is performed using an immunoassay.

10. The method of claim 8, wherein at least one of steps (a)-(c) is performed on ice or at less than 4° C.

11. The method of claim 8, wherein the mild detergent conditions comprise the use of a buffer that contains approximately I % NP-40 or equivalent non-ion detergent.

12. The method of claim 8, wherein the E-cadherin protein or a fragment thereof is fused to a tag.

13. The method of claim 12, wherein the tag is GST, His tag or FLAG.

14. A method of determining the amount of uncomplexed β-catenin in a frozen tissue sample, comprising
(a) preparing a lysate of the frozen sample under mild-detergent conditions,
(b) isolating βj3-catenin from the lysate using GST-E-cadherin beads and
(c) detecting the amount of the isolated β-catenin via immunoassay.

15. A method of determining whether a Wnt signaling is activated in a tumor comprising comparing the level of Axin2 expression in the tumor cells to the level of Axin2 expression in non-tumor normal adjacent tissue cells of the same tissue, wherein an increase in Axin2 expression in the tumor cells as compared to non-tumor normal adjacent tissue cells indicates that the Wnt signaling is activated in the tumor.

16. The method of claim 15, wherein Axin2 expression is determined by RT-PCR or expression RNA profiling.

17. A method for cancer prognosis comprising determining whether canonical Wnt signaling is activated in a tumor, wherein activated canonical Wnt signaling indicates a more aggressive tumor phenotype.

18. The method of claim 17, wherein the canonical Wnt signaling is autocrine Wnt signaling.

19. The method of claim 17, wherein activation of the canonical Wnt signaling is determined using the method of claim 1.

20. The method of claim 17, wherein the tumor does not have genetic alterations of β-catenin or APC.

21. The method of claim 20, wherein the tumor does not have genetic alterations of β-catenin and APC.

22. The method of claim 17, wherein the tumor is Stage I Non-Small Cell Lung Cancer (NSCLC).

23. The method of claim 17, wherein the tumor is selected from the group consisting of lung tumors, sarcomas, brain tumors, breast carcinomas, and ovarian carcinomas.

24.-48. (canceled)

49. A method for identifying whether a tumor would respond to a therapy targeted against activated canonical Wnt signaling comprising determining whether the canonical Wnt signaling is activated in the tumor using the method of claim 1.
